(19) United States
(12) Patent Application Publication
(10) Pub. No.: US 2017/0218407 A1
(43) Pub. Date: Aug. 3, 2017

(54) Biological and Chemical Process Utilizing Chemoautotrophic Microorganisms for the Chemosynthetic Fixation of Carbon Dioxide and/or Other Inorganic Carbon Sources into Organic Compounds and the Generation of Additional Useful Products

(71) Applicant: Kiverdi, Inc., Hayward, CA (US)

(72) Inventor: John S. Reed, Hayward, CA (US)

(21) Appl. No.: 15/485,173

(22) Filed: Apr. 11, 2017

Related U.S. Application Data
(63) Continuation of application No. 13/508,472, filed on Dec. 4, 2012, now abandoned.
Application No. 13/508,472 is a continuation-in-part of application No. 12/613,550, filed on Nov. 6, 2009, now abandoned.
(60) Provisional application No. 61/111,794, filed on Nov. 6, 2008.

(51) Int. Cl. C12P 7/40; C12P 7/16; C12P 7/08

(52) U.S. Cl. CPC C12P 7/40; C12P 7/08; C12P 7/16

(57) ABSTRACT

The invention described herein presents compositions and methods for a multistep biological and chemical process for the capture and conversion of carbon dioxide and/or other forms of inorganic carbon into organic chemicals including biofuels or other useful industrial, chemical, pharmaceutical, or biomass products. One or more process steps utilizes chemoautotrophic microorganisms to fix inorganic carbon into organic compounds through chemosynthesis. An additional feature described are process steps whereby electron donors used for the chemosynthetic fixation of carbon are generated by chemical or electrochemical means, or are produced from inorganic or waste sources. An additional feature described are process steps for the recovery of useful chemicals produced by the carbon dioxide capture and conversion process, both from chemosynthetic reaction steps, as well as from non-biological reaction steps.

### FIELD OF THE INVENTION

The present invention falls within the technical areas of biofuels, bioremediation, carbon capture, carbon dioxide-to-fuels, carbon recycling, carbon sequestration, energy storage, and renewable/alternative and/or low carbon dioxide emission sources of energy. Specifically the present invention involves in certain aspects a unique use of biocatalysts within a biological and chemical process to fix carbon dioxide and/or other forms of inorganic carbon into organic chemical products through chemosynthesis. In addition certain embodiments of the present invention involve the production of chemical co-products that are co-generated through chemosynthetic reaction steps and/or non-biological reaction steps as part of an overall carbon capture and conversion process. The present invention can enable the effective capture of carbon dioxide from the atmosphere or from a point source of carbon dioxide emissions for the production of liquid transportation fuel and/or other organic chemical products, which can help address greenhouse gas induced climate change and contribute to the domestic production of renewable liquid transportation fuels without any dependence upon agriculture.

### BACKGROUND OF THE INVENTION

The amazing technological and economic progress achieved in the past 100 years has largely been powered by fossil fuels. However the sustainability of this progress is now coming into question, both due to the rise in greenhouses gases caused by fossil fuel combustion, and the increasing scarcity of fossil fuel resources.

Hydrogen which can be generated through a number of different inorganic renewable energy technologies including solar, wind, and geothermal has been proposed as a replacement for hydrocarbon fuels. But hydrogen has its own set of problems including most notably problems with storage. Ironically the best chemical storage medium for hydrogen both in terms of volumetric and gravimetric energy densities is quite possibly hydrocarbons such as gasoline, suggesting that the quest for hydrogen fuel may simply lead full circle back to hydrocarbons.

Biofuels are a promising type of renewable hydrocarbon generally made through the capture and conversion of CO₂ into organic matter by photosynthetic organsims. Since the current transportation fleet and infrastructure is designed for fossil fuels with similar properties to biofuels, it can be more readily be adapted to biofuels, than to inorganic energy storage products such as hydrogen or batteries. A further advantage of biofuels, and hydrocarbons in general, is that they have some of the highest volumetric and gravimetric energy densities found for any form of chemical energy storage—substantially higher than that achieved with current lithium battery and hydrogen storage technologies. However, biofuels produced through photosynthesis have their own set of problems.

Most biofuel currently produced relies on agriculture. The heavy requirements of large scale agricultural biofuel projects for arable land, fresh water, and other resources required for plant growth have been blamed for rapidly increasing food prices and loss of natural habitat [The Price of Biofuels: The Economics Behind Alternative Fuels, Technology Review, January/February 2008].

As an alternative to higher order plants, photosynthetic microorganisms such as algae and cyanobacteria are being looked at for applications converting CO₂ into biofuels or other organic chemicals [Sheehan et al, 1998, “A Look Back at the U.S. Department of Energy's Aquatic Species Program—Biodiesel from Algae”].Algal and cyanobacterial technologies benefit from relatively high growth rates, far surpassing higher order plants in their rate of carbon fixation per unit standing biomass. In one promising application of algal technology a high rate of carbon fixation and biomass production is achieved by directing a concentrated stream of CO₂, such as is emitted from industrial point sources, through algae containing bioreactors [Bayless et al. U.S. Pat. No. 6,667,171].

Technologies based on photosynthetic microbes share the drawback common to all photosynthetic systems in that carbon fixation only happens with light exposure. If the light level is deficient, an algal system can actually become a net producer of CO₂ emissions. A bioreactor or pond used to grow photosynthetic microbes such as algae must have a high surface area to volume ratio in order to allow each cell to receive enough light for carbon fixation and cell growth. Otherwise light blockage by cells on the surface will leave cells located towards the center of the volume in darkness—turning them into net CO₂ emitters. This high surface area to volume ratio needed for efficient implementation of the algal and cyanobacterial technologies generally results in either a large land footprint (ponds) or high material costs (bioreactors). The types of materials that can be used in algal bioreactor construction is limited by the requirement that walls lying between the light source and the algal growth environment need to be transparent. This requirement restricts the use of construction materials that would normally be preferred for use in large scale projects such as concrete, steel and earthworks.

In addition to the biological CO₂ fixation processes that have been discussed, there are also fully chemical processes for fixing CO₂ to organic compounds (LBNL Helios; LANL Green Freedom; Sandia Sunshine to Petrol; PARC). The fully chemical technologies are currently hindered by the catalysts that are needed for the relatively complicated reaction of CO₂ to fixed carbon, especially C₂ and longer hydrocarbons.

Chemoautotrophic microorganisms are known that catalyzing the carbon fixation reaction without photosynthesis. The chemosynthetic reactions performed by chemoautotrophs for the fixation of CO₂, and other forms of inorganic carbon, to organic compounds, is powered by potential energy stored in inorganic chemicals, rather than by the radiant energy of light [Shively et al, 1998; Smith et al, 1967; Hugler et al, 2005; Hugker et al., 2005; Scott and Cavanaugh, 2007]. Carbon fixing biochemical pathways that occur in chemoautotrophs include the reductive tricarboxylic acid cycle, the Calvin-Benson-Bassham cycle [Jessup Shively, Geertje van Kaulen, Wim Meijer, Annu. Rev. Microbiol., 1998, 191-230], and the Wood-Ljungdahl pathway [Ljungdahl, 1986; Gottschalk, 1989; Lee, 2008; Fischer, 2008].

Prior work is known relating to certain applications of chemoautotrophic microorganisms in the capture and conversion of CO₂ gas to fixed carbon [U.S. Pat. No. 4,596,778 “Single cell protein from sulfur energy sources” Hitzman, Jun. 24, 1986], [U.S. Pat. No. 4,859,588 “Production of a single cell protein”, Sublette Aug. 22, 1989], [U.S. Pat. No. 5,593,886 “Clostridium strain which produces acetic acid from waste gases Gaddy”, Jan. 14, 1997], [U.S. Pat. No. 5,989,513 “Biologically assisted process for treating sour gas at high pH”, Rai Nov. 23, 1999]. However, each of these conventional approaches have suffered shortcomings that have limited the effectiveness, economic feasibility, practicality and commercial adoption of the described processes. The present invention in certain aspects addresses one or more of the aforementioned shortcomings.

Chemoautotrophic microorganisms have also been used to biologically convert syngas into C₂ and longer organic compounds including acetic acid and acetate, and biofuels such as ethanol and butanol [Gaddy, 2007; Lewis, 2007; Heiskanen, 2007; Worden, 1991; Klasson, 1992; Ahmed, 2006; Cotter, 2008; Piccolo, 2008, Wei, 2008]; however, in such approaches the feedstock is strictly limited to fixed carbon (either biomass or fossil fuel), which is gasified and then biologically converted to another form of fixed carbon—biofuel, and the carbon source and energy source utilized in the process come from the same process input, either biomass or fossil fuel, and are completely intermixed within the syngas in the form of H₂, CO, and CO₂. The present inventors have recognized in the context of the present invention that a need exists for processes that do not require any fixed carbon feedstock, only CO₂ and/or other forms of inorganic carbon and/or utilize a carbon source and energy source that are derived from separate process inputs.

### SUMMARY OF THE INVENTION

In response to a need in the art that the inventors have recognized in making the invention, a novel combined biological and chemical process for the capture and conversion of inorganic carbon to organic compounds that uses chemosynthetic microorganisms for carbon fixation and that is designed to couple the efficient production of high value organic compounds such as liquid hydrocarbon fuel with the capture of CO₂ emissions, making carbon capture a revenue generating process is described.

Described herein are biological and chemical processes for the capture and conversion of carbon dioxide and/or other sources of inorganic carbon, into organic compounds comprising: introducing carbon dioxide gas, either alone and/or dissolved in a mixture or solution further comprising carbonate ion and/or bicarbonate ion, and/or introducing inorganic carbon contained in a solid phase into an environment suitable for maintaining chemoautotrophic organisms and/or chemoautotroph cell extracts; and fixing the carbon dioxide and/or inorganic carbon into organic compounds within the environment via at least one chemosynthetic carbon fixing reaction utilizing obligate and/or facultative chemoautotrophic microorganisms and/or cell extracts containing enzymes from chemoautotrophic microorganisms; wherein where the chemosynthetic carbon fixing reaction is driven by chemical and/or electrochemical energy provided by electron donors and electron acceptors that have been generated chemically and/or electrochemically and/or or are introduced into the environment from at least one source external to the environment.

The carbon source may be separated from the energy source in certain embodiments of the present invention which enables it to function as a far more general energy conversion technology than syngas to liquid fuel conversions. This is because the electron donors used in the present invention can be generated from a wide array of different CO₂-free energy sources, both conventional and alternative, while for syngas conversions to biofuel, all the energy stored in the biofuel is ultimately derived from photosynthesis (with additional geochemical energy in the case of fossil fuel feedstock).

The present invention, in certain embodiments, provides compositions and methods for the capture of carbon dioxide from carbon dioxide-containing gas streams and/or atmospheric carbon dioxide or carbon dioxide in dissolved, liquefied or chemically-bound form through a chemical and biological process that utilizes obligate or facultative chemoautotrophic microorganisms and particularly chemolithoautotrophic organisms, and/or cell extracts containing enzymes from chemoautotrophic microorganisms in one or more carbon fixing process steps. The present invention, in certain embodiments, provides compositions and methods for the recovery, processing, and use of the chemical products of chemosynthetic reactions performed by chemoautotrophs to fix inorganic carbon into organic compounds. The present invention, in certain embodiments, provides compositions and methods for the generation, processing and delivery of chemical nutrients needed for chemosynthesis and maintenance of chemoautotrophic cultures, including but not limited to the provision of electron donors and electron acceptors needed for chemosynthesis. The present invention, in certain embodiments, provides compositions and methods for the maintenance of an environment conducive for chemosynthesis and chemoautotrophic growth, and the recovery and recycling of unused chemical nutrients and process water.

The present invention, in certain embodiments, provides compositions and methods for chemical process steps that occur in series and/or in parallel with the chemosynthetic reaction steps that: convert unrefined raw input chemicals to more refined chemicals that are suited for supporting the chemosynthetic carbon fixing step; that convert energy inputs into a chemical form that can be used to drive chemosynthesis, and specifically into chemical energy in the form of electron donors and electron acceptors; that direct inorganic carbon captured from industrial or atmospheric or aquatic sources to the carbon fixation steps of the process under conditions that are suitable to support chemosynthetic carbon fixation; that further process the output products of the chemosynthetic carbon fixation steps into a form suitable for storage, shipping, and sale, and/or safe disposal in a manner that results in a net reduction of gaseous CO₂ released into the atmosphere. The fully chemical process steps combined with the chemosynthetic carbon fixation steps constitute the overall carbon capture and conversion process of certain embodiments of the present invention. The present invention, in certain embodiments, utilizes the integration of chemoautotrophic microorganisms into a chemical process stream as a biocatalyst, as compared to other lifeforms. This unique capability arises from the fact that chemoautotrophs naturally act at the interface of biology and chemistry through their chemosynthetic lifestyle.

One feature of certain embodiments of the present invention is the inclusion of one or more process steps within a chemical process for the capture of inorganic carbon and conversion to fixed carbon products, that utilize chemoautotrophic microorganisms and/or enzymes from chemoautotrophic microorganisms as a biocatalyst for the fixation of carbon dioxide in carbon dioxide-containing gas streams or the atmosphere or water and/or dissolved or solid forms of inorganic carbon, into organic compounds. In these process steps carbon dioxide containing flue gas, or process gas, or air, or inorganic carbon in solution as dissolved carbon dioxide, carbonate ion, or bicarbonate ion including aqueous solutions such as sea water, or inorganic carbon in solid phases such as but not limited to carbonates and bicarbonates, may be pumped or otherwise added to a suitable environment, such as a vessel or enclosure containing nutrient media and chemoautotrophic microorganisms. In these process steps chemoautotrophic microorganisms perform chemosynthesis to fix inorganic carbon into organic compounds using the chemical energy stored in one or more types of electron donor pumped or otherwise provided to the nutrient media including but not limited to one of more of the following: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate or calcium thiosulfate ; sulfides such as hydrogen sulfide; sulfites; thionate; thionite; transition metals or their sulfides, oxides, chalcogenides, halides, hydroxides, oxyhydroxides, sulfates, or carbonates, in soluble or solid phases; as well as valence or conduction electrons in solid state electrode materials. The electron donors are oxidized by electron acceptors in the chemosynthetic reaction. Electron acceptors that may be used at the chemosynthetic reaction step include but are not limited to one or more of the following: carbon dioxide, ferric iron or other transition metal ions, nitrates, nitrites, oxygen, sulfates, or holes in solid state electrode materials.

The chemosynthetic reaction step or steps of certain inventive processes wherein carbon dioxide and/or inorganic carbon is fixed into organic carbon in the form of organic compounds and biomass can be performed in aerobic, microaerobic, anoxic, anaerobic, or facultative conditions. A facultative environment is considered to be one where the water column is stratified into aerobic layers and anaerobic layers. The oxygen level maintained spatially and temporally in the system will depend upon the chemoautotrophic species used, and the desired chemosynthesis reactions to be performed.

An additional feature of certain embodiments of the present invention regards the source, production, or recycling of the electron donors used by the chemoautotrophic microorganisms to fix carbon dioxide into organic compounds. The electron donors used for carbon dioxide capture and carbon fixation can be produced or recycled in the present invention electrochemically or thermochemically using power from a number of different renewable and/or low carbon emission energy technologies including but not limited to: photovoltaics, solar thermal, wind power, hydroelectric, nuclear, geothermal, enhanced geothermal, ocean thermal, ocean wave power, tidal power. The electron donors can also be of mineralogical origin including but not limited to reduced S and Fe containing minerals. The present invention enables the use of a largely untapped source of energy—inorganic geochemical energy. The electron donors used in the present invention can also be produced or recycled through chemical reactions with hydrocarbons that may or may not be a non-renewable fossil fuel, but where said chemical reactions produce low or zero carbon dioxide gas emissions. Such electron donor generating chemical reactions that can be used as steps in the process certain embodiments of the present invention include but are not limited to: the thermochemical reduction of sulfate reaction or TSR [Evaluating the Risk of Encountering Non-hydrocarbon Gas Contaminants (CO₂, N₂, H₂S) Using Gas Geochemistry, www.gaschem.com/evalu.html] or the Muller-Kuhne reaction; the reduction of metal oxides including iron oxide, calcium oxide, and magnesium oxide. The reaction formula for TSR is CaSO₄+CH₄→CaCO₃+H₂O+H₂S. In this case the electron donor product that can be used by chemoautotrophic microorganisms for CO₂ fixation is hydrogen sulfide. The solid carbonate product also formed can be easily sequestered resulting in no release of carbon dioxide into the atmosphere. There are similar reactions reducing sulfate to sulfide that involve longer chain hydrocarbons [Changtao Yue, Shuyuan Li, Kangle Ding, Ningning Zhong, Thermodynamics and kinetics of reactions between C₁-C₃ hydrocarbons and calcium sulfate in deep carbonate reservoirs, Geochem. Jour., 2006, 87-94].

An additional feature of certain embodiments of the present invention regards the formation and recovery of useful organic and/or inorganic chemical products from the chemosynthetic reaction step or steps including but not limited to one ore more of the following: acetic acid, other organic acids and salts of organic acids, ethanol, butanol, methane, hydrogen, hydrocarbons, sulfuric acid, sulfate salts, elemental sulfur, sulfides, nitrates, ferric iron and other transition metal ions, other salts, acids or bases. These chemical products can be applied to uses including but not limited to one or more of the following: as a fuel; as a feedstock for the production of fuels; in the production of fertilizers; as a leaching agent for the chemical extraction of metals in mining or bioremediation; as chemicals reagents in industrial or mining processes.

An additional feature of certain embodiments of the present invention regards the formation and recovery of biochemicals and/or biomass from the chemosynthetic carbon fixation step or steps. These biochemical and/or biomass products can have applications including but not limited to one or more of the following: as a biomass fuel for combustion in particular as a fuel to be co-fired with fossil fuels such as coal in pulverized coal powered generation units; as a carbon source for large scale fermentations to produce produce various chemicals including but not limited to commercial enzymes, antibiotics, amino acids, vitamins, bioplastics, glycerol, or 1,3-propanediol; as a nutrient source for the growth of other microbes or organisms; as feed for animals including but not limited to cattle, sheep, chickens, pigs, or fish; as feed stock for alcohol or other biofuel fermentation and/or gasification and liquefaction processes including but not limited to direct liquefaction, Fisher Tropsch processes, methanol synthesis, pyrolysis, transesterification, or microbial syngas conversions, for the production of liquid fuel; as feed stock for methane or biogas production; as fertilizer; as raw material for manufacturing or chemical processes such as but not limited to the production of biodegradable/biocompatible plastics; as sources of pharmaceutical, medicinal or nutritional substances; soil additives and soil stabilizers.

An additional feature of certain embodiments of the present invention regards using modified chemoautotrophic microorganisms in the chemosynthesis process step/steps such that a superior quantity and/or quality of organic compounds, biochemicals, or biomass is generated through chemosynthesis. The chemoautotrophic microbes used in these steps may be modified through artificial means including but not limited to accelerated mutagenesis (e.g. using ultraviolet light or chemical treatments), genetic engineering or modification, hybridization, synthetic biology or traditional selective breeding.

Other advantages and novel features of the present invention will become apparent from the following detailed description of various non-limiting embodiments of the invention when considered in conjunction with the accompanying figures. All publications, patent applications and patents mentioned in the text are incorporated by reference in their entirety. In cases where the present specification and a document incorporated by reference include conflicting and/or inconsistent disclosure, the present specification shall control.

### BRIEF DESCRIPTION OF THE FIGURES

Non-limiting embodiments of the present invention will be described by way of example with reference to the accompanying figures, which are schematic and are not intended to be drawn to scale. For purposes of clarity, not every component is labeled in every figure, nor is every component of each embodiment of the invention shown where illustration is not necessary to allow those of ordinary skill in the art to understand the invention. In the figures:

FIG. 1 is a general process flow diagram for one embodiment of this invention for a carbon capture and fixation process;

FIG. 2 is process flow diagram for another embodiment of the present invention with capture of CO₂ performed by hydrogen oxidizing chemoautotrophs resulting in the production of ethanol;

FIG. 3 shows the mass balance calculated for the embodiment of FIG. 2 reacting CO₂ with H₂ to produce ethanol;

FIG. 4 shows the enthalpy flow calculated for the embodiment of FIG. 2 reacting CO₂ with H₂ to produce ethanol;

FIG. 5 shows the energy balance calculated for the embodiment of FIG. 2 reacting CO₂ with H₂ to produce ethanol;

FIG. 6. is a process flow diagram for the capture of CO₂ by sulfur oxidizing chemoautotrophs and production of biomass and sulfuric acid, according to one embodiment;

FIG. 7. is a process flow diagram for the capture of CO₂ by sulfur oxidizing chemoautotrophs and production of biomass and sulfuric acid through the chemosynthetic reaction and calcium carbonate via the Muller-Kuhne reaction, according to one embodiment;

FIG. 8 is a process flow diagram for the capture of CO₂ by sulfur oxidizing chemoautotrophs and production of biomass and calcium carbonate and recycling of thiosulfate electron donor via the Muller-Kuhne reaction, according to one embodiment; FIG. 9 is a process flow diagram for the capture of CO₂ by sulfur and iron oxidizing chemoautotrophs and production of biomass and sulfuric acid using an insoluble source of electron donors, according to one embodiment;

FIG. 10 is a process flow diagram for the capture of CO₂ by sulfur and hydrogen oxidizing chemoautotrophs and production of biomass, sulfuric acid, and ethanol using an insoluble source of electron donors, according to one embodiment; and

FIG. 11 is a process flow diagram for the capture of CO₂ by iron and hydrogen oxidizing chemoautotrophs and production of biomass, ferric sulfate, carbonate and ethanol using coal or another hydrocarbon to generate electron donors in a process that does not emit gaseous CO₂ emissions, according to one embodiment.

### DETAILED DESCRIPTION

The present invention provides, in certain embodiments, compositions and methods for the capture and fixation of carbon dioxide from carbon dioxide-containing gas streams and/or atmospheric carbon dioxide or carbon dioxide in liquefied or chemically-bound form through a chemical and biological process that utilizes obligate or facultative chemoautotrophic microorganisms and particularly chemolithoautotrophic organisms, and/or cell extracts containing enzymes from chemoautotrophic microorganisms in one or more process steps. Cell extracts include but are not limited to: a lysate, extract, fraction or purified product exhibiting chemosynthetic enzyme activity that can be created by standard methods from chemoautotrophic microorganisms. In addition the present invention, in certain embodiments, provides compositions and methods for the recovery, processing, and use of the chemical products of chemosynthetic reaction step or steps performed by chemoautotrophs to fix inorganic carbon into organic compounds. Finally the present invention, in certain embodiments, provides compositions and methods for the production and processing and delivery of chemical nutrients needed for chemosynthesis and chemoautotrophic growth, and particularly electron donors and acceptors to drive the chemosynthetic reaction; compositions and methods for the maintenance of a environment conducive for chemosynthesis and chemoautotrophic growth; and compositions and methods for the removal of the chemical products of chemosynthesis from the chemoautotrophic growth environment and the recovery and recycling of unused of chemical nutrients.

The genus of chemoautotrophic microorganisms that can be used in one or more process steps of the present invention include but are not limited to one or more of the following: Acetoanaerobium sp., Acetobacterium sp., Acetogenium sp., Achromobacter sp., Acidianus sp., Acinetobacter sp., Actinomadura sp., Aeromonas sp., Alcaligenes sp., Alcaligenes sp., Arcobacter sp., Aureobacterium sp., Bacillus sp., Beggiatoa sp., Butyribacterium sp., Carboxydothermus sp., Clostridium sp., Comamonas sp., Dehalobacter sp., Dehalococcoide sp., Dehalospirillum sp., Desulfobacterium sp., Desulfomonile sp., Desulfotomaculum sp., Desulfovibrio sp., Desulfurosarcina sp., Ectothiorhodospira sp., Enterobacter sp., Eubacterium sp., Ferroplasma sp., Halothibacillus sp., Hydrogenobacter sp., Hydrogenomonas sp., Leptospirillum sp., Metallosphaera sp., Methanobacterium sp., Methanobrevibacter sp., Methanococcus sp., Methanosarcina sp., Micrococcus sp., Nitrobacter sp., Nitrosococcus sp., Nitrosolobus sp., Nitrosomonas sp., Nitrosospira sp., Nitrosovibrio sp., Nitrospina sp., Oleomonas sp., Paracoccus sp., Peptostreptococcus sp., Planctomycetes sp., Pseudomonas sp., Ralstonia sp., Rhodobacter sp., Rhodococcus sp., Rhodocyclus sp., Rhodomicrobium sp., Rhodopseudomonas sp., Rhodospirillum sp., Shewanella sp., Streptomyces sp., Sulfobacillus sp., Sulfolobus sp., Thiobacillus sp., Thiomicrospira sp, Thioploca sp., Thiosphaera sp., Thiothrix sp. Also chemoautotrophic microorganisms that are generally categorized as sulfur-oxidizers, hydrogen-oxidizers, iron-oxidizers, acetogens, methanogens, as well as a consortiums of microorganisms that include chemoautotrophs.

The different chemoautotrophs that can be used in the present invention may be native to a range environments including but not limited to hydrothermal vents, geothermal vents, hot springs, cold seeps, underground aquifers, salt lakes, saline formations, mines, acid mine drainage, mine tailings, oil wells, refinery wastewater, coal seams, the deep sub-surface, waste water and sewage treatment plants, geothermal power plants, sulfatara fields, soils. They may or may not be extremophiles including but not limited to thermophiles, hyperthermophiles, acidophiles, halophiles, and psychrophiles.

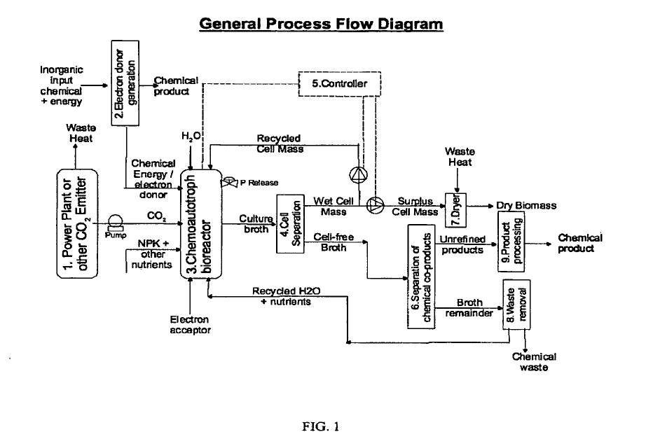

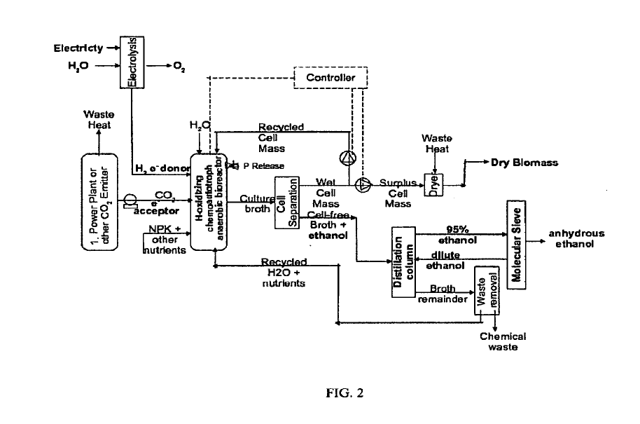

FIG. 1 illustrates the general process flow diagram for certain embodiments of the present invention that have a process step for the generation of electron donors suitable for supporting chemosynthesis from an energy input and raw inorganic chemical input; followed by recovery of chemical products from the electron donor generation step; delivery of generated electron donors along with electron acceptors, water, nutrients, and CO₂ from a point industrial flue gas source, into chemosynthetic reaction step or steps that make use of chemoautotrophic microorganisms to capture and fix carbon dioxide, creating chemical and biomass co-products through chemosynthetic reactions; followed by process steps for the recovery of both chemical and biomass products from the process stream; and recycling of unused nutrients and process water, as well as cell mass needed to maintain the chemoautotrophic culture back into the chemosynthetic reaction steps. In the embodiment illustrated in FIG. 1, the CO₂ containing flue gas is captured from a point source or emitter. Electron donors needed for chemosynthesis may be generated from input inorganic chemicals and energy. The flue gas is pumped through bioreactors containing chemoautotrophs along with electron donors and acceptors to drive chemosynthesis and a medium suitable to support a chemoautotrophic culture and carbon fixation through chemosynthesis. The cell culture may be continuously flowed into and out of the bioreactors. After the cell culture leaves the bioreactors the cell mass is separated from the liquid medium. Cell mass needed to replenish the cell culture population at a functional or an optimal level is recycled back into the bioreactor. Surplus cell mass may be dried to form a dry biomass product. Following the cell separation step chemical products of the chemosynthetic reaction may be removed from the process flow and recovered. Then any undesirable waste products that might be present may be removed. Following this, in the illustrated embodiment, the liquid medium and any unused nutrients are recycled back into the bioreactors. Many of the reduced inorganic chemicals upon which chemoautotrophs grow (e.g. H₂, H₂S, ferrous iron, ammonium, Mn²⁺) can be readily produced using electrochemical and/or thermochemical processes known in the art of chemical engineering that may optionally be powered by a variety carbon dioxide emission-free or low-carbon emission and/or renewable sources of power including wind, hydroelectric, nuclear, photovoltaics, or solar thermal.

Certain embodiments of the present invention use carbon dioxide emission-free or low-carbon emission and/or renewable sources of power in the production of electron donors including but not limited to one or more of the following: photovoltaics, solar thermal, wind power, hydroelectric, nuclear, geothermal, enhanced geothermal, ocean thermal, ocean wave power, tidal power. In certain embodiments of the present invention that draw upon carbon dioxide emission-free or low-carbon emission and/or renewable sources of power in the production of electron donors, chemoautotrophs function as biocatalysts for the conversion of renewable energy into liquid hydrocarbon fuel, or high energy density organic compounds generally, with CO₂ captured from flue gases, or from the atmosphere, or ocean serving as a carbon source. These embodiments of the present invention can provide renewable energy technologies with the capability of producing a transportation fuel having significantly higher energy density than if the renewable energy sources are used to produce hydrogen gas—which must be stored in relatively heavy storage systems (e.g. tanks or storage materials)—or if it is used to charge batteries which have relatively low energy density. Additionally the liquid hydrocarbon fuel product of certain embodiments of the present invention may be more compatible with the current transportation infrastructure compared to these other energy storage options. The ability of chemoautotrophs to use inorganic sources of chemical energy also enables the conversion of inorganic carbon into liquid hydrocarbon fuels using non-hydrocarbon mineralogical sources of chemical energy, i.e. reduced inorganic minerals (such as hydrogen sulfide, pyrite), which represent a largely untapped store of geochemical energy. Hence certain embodiments of the present invention use mineralogical sources of chemical energy which are pre-processed ahead of the chemosynthetic reaction steps into a form of electron donor and method of electron donor delivery that is suitable or optimal for supporting chemoautotrophic carbon fixation.

The position of the process step or steps for the generation of electron donors in the general process flow of the present invention is illustrated in FIG. 1 by the box 2. labeled “Electron Donor Generation”. Electron donors produced in the present invention using electrochemical and/or thermochemical processes known in the art of chemical engineering and/or generated from natural sources include but are not limited to one or more of the following: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate or calcium thiosulfate ; sulfides such as hydrogen sulfide; sulfites; thionate; thionite; transition metals or their sulfides, oxides, chalcogenides, halides, hydroxides, oxyhydroxides, sulfates, or carbonates, in soluble or solid phases; as well as valence or conduction electrons in solid state electrode materials.

Certain embodiments of the present invention use molecular hydrogen as electron donor. Hydrogen electron donor may be generated by methods known in to art of chemical and process engineering including but not limited to more or more of the following: through electrolysis of water including but not limited to approaches using Proton Exchange Membranes (PEM), liquid electrolytes such as KOH, high-pressure electrolysis, high temperature electrolysis of steam (HTES); thermochemical splitting of water through methods including but not limited to the iron oxide cycle, cerium(IV) oxide-cerium(III) oxide cycle, zinc zinc-oxide cycle, sulfur-iodine cycle, copper-chlorine cycle, calcium-bromine-iron cycle, hybrid sulfur cycle; electrolysis of hydrogen sulfide; thermochemical splitting of hydrogen sulfide; other electrochemical or thermochemical processes known to produce hydrogen with low- or no-carbon dioxide emissions including but not limited to: carbon capture and sequestration enabled methane reforming; carbon capture and sequestration enabled coal gasification; the Kvaerner-process and other processes generating a carbon-black product; carbon capture and sequestration enabled gasification or pyrolysis of biomass; and the half-cell reduction of H+ to H₂ accompanied by the half-cell oxidization of electron sources including but not limited to ferrous iron (Fe²⁺) oxidized to ferric iron (Fe³⁺) or the oxidation of sulfur compounds whereby the oxidized iron or sulfur can be recycled to back to a reduced state through additional chemical reaction with minerals including but not limited to metal sulfides, hydrogen sulfide, or hydrocarbons.

Certain embodiments of the present invention utilize electrochemical energy stored in solid-state valence or conduction electrons within an electrode or capacitor or related devices, alone or in combination with chemical electron donors and/or electron mediators to provide the chemoautotrophs electron donors for the chemosynthetic reactions by means of direct exposure of said electrode materials to the chemoautotrophic culturing environment.

Certain embodiments of the present invention that use electrical power for the generation of electron donors, receive the electrical power from carbon dioxide emission-free or low-carbon emission and/or renewable sources of power in the production of electron donors including but not limited to one or more of the following: photovoltaics, solar thermal, wind power, hydroelectric, nuclear, geothermal, enhanced geothermal, ocean thermal, ocean wave power, tidal power.

A feature of certain embodiments of the present invention regards the production, or recycling of electron donors generated from mineralogical origin including but not limited electron donors generated from reduced S and Fe containing minerals. Hence the present invention, in certain embodiments, enables the use of a largely untapped source of energy—inorganic geochemical energy. There are large deposits of sulfide minerals that could be used for this purpose located in all the continents and particularly in regions of Africa, Asia, Australia, Canada, Eastern Europe, South America, and the USA. Geological sources of S and Fe such as hydrogen sulfide and pyrite, constitute a relatively inert and sizable pool of S and Fe in the respective natural cycles of sulfur and iron. Sulfides can be found in igneous rocks as well as sedimentary rocks or conglomerates. In some cases sulfides constitute the valuable part of a mineral ore, in other cases such as with coal, oil, methane, or precious metals the sulfides are considered to be impurities. In the case of fossil fuels, regulations such as Clean Air Act require the removal of sulfur impurities to prevent sulfur dioxide emissions. The use of inorganic geochemical energy facilitated by certain embodiments of the present invention appears to be largely unprecedented, and hence the present invention represents a novel alternative energy technology.

The electron donors used in the present invention may be refined from natural mineralogical sources which include but are not limited to one or more of the following: elemental Fe⁰; siderite (FeCO₃); magnetite (Fe₃O₄); pyrite or marcasite (FeS₂), pyrrhotite (Fe₍₁₋ₓ₎S (x=0 to 0.2), pentlandite (Fe,Ni)₉S₈, violarite (Ni₂FeS₄), bravoite (Ni,Fe)S₂, arsenopyrite (FeAsS), or other iron sulfides; realgar (AsS); orpiment (As₂S₃); cobaltite (CoAsS); rhodochrosite (MnCO₃); chalcopyrite (CuFeS₂), bornite (Cu₅FeS₄), covellite (CuS), tetrahedrite (Cu₈Sb₂S₇), enargite (Cu₃AsS₄), tennantite (Cu₁₂As₄.S₁₃), chalcocite (Cu₂S), or other copper sulfides; sphalerite (ZnS), marmatite (ZnS), or other zinc sulfides; galena (PbS), geocronite (Pb₅(Sb,As₂)S₈), or other lead sulfides; argentite or acanthite (Ag₂S); molybdenite (MoS₂); millerite (NiS), polydymite (Ni₃S₄) or other nickel sulfides; antimonite (Sb₂S₃); Ga₂S₃; CuSe; cooperite (PtS); laurite (RuS₂); braggite (Pt,Pd,Ni)S; FeCl₂.

The generation of electron donor from natural mineralogical sources includes a preprocessing step in certain embodiments of the present invention which can include but is not limited to comminuting, crushing or grinding mineral ore to increase the surface area for leaching with equipment such as a ball mill and wetting the mineral ore to make a slurry. In these embodiments of the present invention where electron donors are generated from natural mineral sources, it may be advantageous if particle size is controlled so that the sulfide and/or other reducing agents present in the ore may be concentrated by methods known to the art including but not limited to: flotation methods such as dissolved air flotation or froth flotation using flotation columns or mechanical flotation cells; gravity separation; magnetic separation; heavy media separation; selective agglomeration; water separation; or fractional distillation. After the production of crushed ore or slurry, the particulate matter in the leachate or concentrate may be separated by filtering (e.g. vacuum filtering), settling, or other well known techniques of solid/liquid separation, prior to introducing the electron donor containing solution to the chemoautotrophic culture environment. In addition anything toxic to the chemoautotrophs that is leached from the mineral ore may be removed prior to exposing the chemoautotrophs to the leachate. The solid left after processing the mineral ore may be concentrated with a filter press, disposed of, retained for further processing, or sold depending upon the mineral ore used in the particular embodiment of the invention.

The electron donors in the present invention may also be refined from pollutants or waste products including but are not limited to one or more of the following: process gas; tail gas; enhanced oil recovery vent gas; biogas; acid mine drainage; landfill leachate; landfill gas; geothermal gas; geothermal sludge or brine; metal contaminants; gangue; tailings; sulfides; disulfides; mercaptans including but not limited to methyl and dimethyl mercaptan, ethyl mercaptan; carbonyl sulfide; carbon disulfide; alkanesulfonates; dialkyl sulfides; thiosulfate; thiofurans; thiocyanates; isothiocyanates; thioureas; thiols; thiophenols; thioethers; thiophene; dibenzothiophene; tetrathionate; dithionite; thionate; dialkyl disulfides; sulfones; sulfoxides; sulfolanes; sulfonic acid; dimethyl sulfoniopropionate; sulfonic esters; hydrogen sulfide; sulfate esters; organic sulfur; sulfur dioxide and all other sour gases.

In addition to mineralogical sources, electron donors are produced or recycled in certain embodiments of the present invention through chemical reactions with hydrocarbons that may be of fossil origin, but which are used in chemical reactions producing low or zero carbon dioxide gas emissions. These reactions include thermochemical and electrochemical processes. Such chemical reactions that are used in these embodiments of the present invention include but are not limited to: the thermochemical reduction of sulfate reaction or TSR and the Muller-Kuhne reaction; methane reforming-like reactions utilizing metal oxides in place of water such as but not limited to iron oxide, calcium oxide, or magnesium oxide whereby the hydrocarbon is reacted to form solid carbonate with little or no emissions of carbon dioxide gas along with hydrogen electron donor product.

The reaction formula for TSR is CaSO₄+CH₄→CaCO₃+H₂O+H₂S. In this case the electron donor product that can be used by chemoautotrophic microorganisms for CO₂ fixation is hydrogen sulfide (H₂S) or the H₂S can by further reacted electrochemically or thermochemically to produce H₂S electron donor using processes known in the art of chemical engineering. The solid carbonate product (CaCO₃) also formed in the TSR can be easily sequestered and applied to a number of different applications, resulting in essentially no release of carbon dioxide into the atmosphere. There are similar reactions reducing sulfate to sulfide that involve longer chain hydrocarbons including short- and long-chain alkanes and complex aliphatic and aromatic compounds [Changtao Yue, Shuyuan Li, Kangle Ding, Ningning Zhong, Thermodynamics and kinetics of reactions between C₁-C₃ hydrocarbons and calcium sulfate in deep carbonate reservoirs, Geochem. Jour., 2006, 87-94].

The Muller-Kuhne reaction formula is 2C+4CaSO₄→2CaO+2CaCO₃+4SO₂. The SO₂ produced can be further reacted with S and a base including but not limited to lime, magnesium oxide, iron oxide, or some other metal oxide to produce an electron donor such as thiosulfate (S₂O₃²⁻) usable by chemoautotrophs. In certain embodiments, the base used in the reaction to form (S₂O₃²⁺) is produced from a carbon dioxide emission-free source such as natural sources of basic minerals including but not limited to calcium oxide, magnesium oxide, olivine containing a metal oxide, serpentine containing a metal oxide, ultramafic deposits containing metal oxides, and underground basic saline aquifers. For embodiments of the present invention using variations of the TSR or Muller-Kuhne, hydrocarbons sources may be utilized which have little or no current economic value such as tar sand or oil shale.

Examples of reactions between metal oxides and hydrocarbons to produce a hydrogen electron donor product and carbonates include but are not limited to 2CH₄+Fe₂O₃+3H₂O →2FeCO₃+7H₂ or CH₄+CaO+2H₂O→CaCO₃+4H₂.

Since reactions like the TSR are exothermic, for embodiments of the present invention that utilize the TSR for electron donor generation heat energy released by the TSR may be recovered using heat exchange methods known in the art of process engineering, to improve the efficiency of the overall process. One embodiment of the invention uses heat released by the TSR as a heat source for maintaining the proper bioreactor temperature or drying the biomass.

In certain embodiments, the generated electron donors are oxidized in the chemosynthetic reaction step or steps by electron acceptors that include but are not limited to one or more of the following: carbon dioxide, ferric iron or other transition metal ions, nitrates, nitrites, oxygen, sulfates, or holes in solid state electrode materials.

The position of the chemosynthetic reaction step or steps in the general process flow of the present invention is illustrated in FIG. 1 by the box 3. labeled “Chemoautotroph bioreactor”.

At each step in the process where chemosynthetic reactions occur one or more types of electron donor and one or more types of electron acceptor may be pumped or otherwise added to the reaction vessel as either a bolus addition, or periodically, or continuously to the nutrient medium containing chemoautotrophic organisms. The chemosynthetic reaction driven by the transfer of electrons from electron donor to electron acceptor fixes inorganic carbon dioxide into organic compounds and biomass.

In certain embodiments of the present invention electron mediators may be included in the nutrient medium to facilitate the delivery of reducing equivalents from electron donors to chemoautotrophic organisms in the presence of electron acceptors and inorganic carbon in order to kinetically enhance the chemosynthetic reaction step. This aspect of the present invention is particularly applicable to embodiments of the present invention using poorly soluble electron donors such as but not limited to H₂ gas or electrons in solid state electrode materials. The delivery of reducing equivalents from electron donors to the chemoautotrophic organisms for the chemosynthetic reaction or reactions can be kinetically and/or thermodynamically enhanced in the present invention through means including but not limited to: the introduction of hydrogen storage materials into the chemoautotrophic culture environment that can double as a solid support media for microbial growth—bringing absorbed or adsorbed hydrogen electron donors into close proximity with the hydrogen-oxidizing chemoautotrophs; the introduction of electron mediators known in the art such as but not limited to cytochromes, formate, methyl-viologen, NAD+/NADH, neutral red (NR), and quinones into the chemoautotrophic culture media; the introduction of electrode materials that can double as a solid growth support media directly into the chemoautotrophic culture environment—bringing solid state electrons into close proximity with the microbes.

The culture broth used in the chemosynthetic steps of certain embodiments of the present invention may be an aqueous solution containing suitable minerals, salts, vitamins, cofactors, buffers, and other components needed for microbial growth, known to those skilled in the art [Bailey and Ollis, Biochemical Engineering Fundamentals, 2nd ed; pp 383-384 and 620-622; McGraw-Hill: New York (1986)]. These nutrients can be chosen to facilitate or maximize chemoautotrophic growth and promote the chemosynthetic enzymatic pathways. Alternative growth environments such as used in the arts of solid state or non-aqueous fermentation may be used in certain embodiments. In certain embodiments that utilize an aqueous culture broth, salt water, sea water, or other non-potable sources of water are used when tolerated by the chemoautotrophic organisms.

The chemosynthetic pathways may be controlled and optimized in certain embodiments of the present invention for the production of chemical products and/or biomass by maintaining specific growth conditions (e.g. levels of nitrogen, oxygen, phosphorous, sulfur, trace micronutrients such as inorganic ions, and if present any regulatory molecules that might not generally be considered a nutrient or energy source). Depending upon the embodiment of the invention the broth may be maintained in aerobic, microaerobic, anoxic, anaerobic, or facultative conditions depending upon the requirements of the chemoautotrophic organisms and the desired products to be created by the chemosynthetic process. A facultative environment is considered to be one having aerobic upper layers and anaerobic lower layers caused by stratification of the water column.

The source of inorganic carbon used in the chemosynthetic reaction process steps of certain embodiments of the present invention includes but is not limited to one or more of the following: a carbon dioxide-containing gas stream that may be pure or a mixture; liquefied CO₂; dry ice; dissolved carbon dioxide, carbonate ion, or bicarbonate ion in solutions including aqueous solutions such as sea water; inorganic carbon in a solid form such as a carbonate or bicarbonate minerals. Carbon dioxide and/or other forms of inorganic carbon may be introduced to the nutrient medium contained in reaction vessels either as a bolus addition or periodically or continuously at the steps in the process where chemosynthesis occurs. In certain embodiments of the present invention, carbon dioxide containing flue gases are captured from the smoke stack at temperature, pressure, and gas composition characteristic of the untreated exhaust, and directed with minimal modification into the reaction vessel(s) where chemosynthesis occurs. Particularly for embodiments where impurities harmful to chemoautotrophic organisms are not present in the flue gas, modification of the flue gas upon entering the reaction vessels may be substantially limited to compression needed to pump the gas through the reactor system and heat exchange needed to lower the gas temperature to one suitable for the microorganisms.

Gases in addition to carbon dioxide that are dissolved into the culture broth of certain embodiments of the present invention may include gaseous electron donors in certain embodiments such as but not limited to hydrogen, carbon monoxide, hydrogen sulfide or other sour gases; and for certain aerobic embodiments of the present invention, oxygen electron acceptor, generally from air (e.g. 20.9% oxygen): The dissolution of these and other gases into solution may be achieved using a system of compressors, flowmeters, and flow valves known to one of skilled in the art of bioreactor scale microbial culturing, that feed into one of more of the following widely used systems for pumping gas into solution: sparging equipment; diffusers including but not limited to dome, tubular, disc, or doughnut geometries; coarse or fine bubble aerators; venturi equipment. In certain embodiments of the present invention surface aeration may also be performed using paddle aerators and the like. In certain embodiments of the present invention gas dissolution is enhanced by mechanical mixing with an impeller or turbine, as well as hydraulic shear devices to reduce bubble size. Following passage through the reactor system holding chemoautotrophic microorganisms which capture the carbon dioxide, the scrubbed flue gas, which is generally comprised primarily of inert gases such as nitrogen, may be released into the atmosphere.

In certain embodiments of the present invention utilizing hydrogen as electron donor, hydrogen gas is fed to the chemoautotrophic bioreactor either by bubbling it through the culture medium, or by diffusing it through a membrane that bounds the culture medium. The latter method may be safer in certain cases, since hydrogen accumulating in the gas phase can potentially create explosive conditions (the range of explosive hydrogen concentrations in air is 4 to 74.5% and may be avoided in certain embodiments of the present invention).

In certain aerobic embodiments of the present invention that require the pumping of air or oxygen into the culture broth in order to maintain oxygenated levels, oxygen bubbles are injected into the broth at an appropriate or optimal diameter for mixing and oxygen transfer. In one exemplary embodiment, the average diameter of the oxygen bubbles is selected to be about 2 mm, which has been found to be optimal in certain cases [Environment Research Journal May/June 1999 pgs. 307-315]. In certain aerobic embodiments of the present invention a process of shearing the oxygen bubbles is used to achieve this bubble diameter as described in U.S. Pat. No. 7,332,077. In certain embodiments, bubble size is controlled to yield values a no larger than 7.5 mm average diameter without substantial slugging.

Additional chemicals to facilitate chemoautotrophic maintenance and growth as known in the art may be added to the culture broth of certain embodiments of the present invention. The concentrations of nutrient chemicals, and particularly the electron donors and acceptors, may be maintained as close as possible to their respective optimal levels for maximum chemoautotrophic growth and/or carbon uptake and fixation and/or production of organic compounds, which varies depending upon the chemoautotrophic species utilized but is known or determinable without undue experimentation to one of skilled in the art of culturing chemoautotrophs.

Along with nutrient levels, the waste product levels, pH, temperature, salinity, dissolved oxygen and carbon dioxide, gas and liquid flow rates, agitation rate, and pressure in the chemoautotrophic culture environment may be controlled in certain embodiments of the present invention as well. The operating parameters affecting chemoautotrophic growth may be monitored with sensors (e.g. dissolved oxygen probe or oxidation-reduction probe to gauge electron donor/acceptor concentrations,), and controlled either manually or automatically based upon feedback from sensors through the use of equipment including but not limited to actuating valves, pumps, and agitators. The temperature of the incoming broth as well as incoming gases may be regulated by unit operations such as but not limited to heat exchangers.

Agitation of the culture broth in certain embodiments of the present invention may be provided for mixing and may be accomplished by equipment including but not limited to: recirculation of broth from the bottom of the container to the top via a recirculation conduit; sparging with carbon dioxide plus in certain embodiments electron donor gas (e.g. H₂ or H₂S), and for certain aerobic embodiments of the present invention oxygen or air as well; a mechanical mixer such as but not limited to an impeller (100-1000 rpm) or turbine.

In certain embodiments, the chemoautotrophic microorganism containing nutrient medium is removed from the chemosynthetic reactors partially or completely, periodically or continuously, and is replaced with fresh cell-free medium to maintain the cell culture in exponential growth phase and/or replenish the depleted nutrients in the growth medium and/or remove inhibitory waste products.

The production of useful chemical products through the chemosynthetic reaction step or steps reacting elecron donors and acceptors to fix carbon dioxide is a feature of certain embodiments of the present invention. These useful chemical products, both organic and inorganic, can include but are not limited to one or more of the following: acetic acid, other organic acids and salts of organic acids, ethanol, butanol, methane, hydrogen, hydrocarbons, sulfuric acid, sulfate salts, elemental sulfur, sulfides, nitrates, ferric iron and other transition metal ions, other salts, acids or bases. Optimizing the production of a desired chemical product of chemosynthesis may be achieved in certain embodiments of the present invention through control of the parameters in the chemoautotrophic culture environment including but not limited to: nutrient levels, waste levels, pH, temperature, salinity, dissolved oxygen and carbon dioxide, gas and liquid flow rates, agitation rate, and pressure

The high growth rate of certain chemoautotrophic species enables them to equal or even surpass the highest rates of carbon fixation, and biomass production per standing unit biomass attainable by photosynthetic microbes. Consequently the production of surplus biomass is a feature of certain embodiments of the present invention. Surplus growth of cell mass may be removed from the system to produce a biomass product, and in order to maintain an optimal microbial population and cell density in the chemoautotrophic culture for continued high carbon capture and fixation rates.

Another feature of certain embodiments of the present invention is the vessels used to contain the chemosynthetic reaction environment in the carbon capture and fixation process. The types of culture vessels that can be used in the present invention to culture and grow the chemoautotrophic bacteria for carbon dioxide capture and fixation are generally known in the art of large scale microbial culturing. These culture vessels, which may be of natural or artificial origin, include but are not limited to: airlift reactors; biological scrubber columns; bioreactors; bubble columns; caverns; caves; cisterns; continuous stirred tank reactors; counter-current, upflow, expanded-bed reactors; digesters and in particular digester systems such as known in the prior arts of sewage and waste water treatment or bioremediation; filters including but not limited to trickling filters, rotating biological contactor filters, rotating discs, soil filters; fluidized bed reactors; gas lift fermenters; immobilized cell reactors; lagoons; membrane biofilm reactors; mine shafts; pachuca tanks; packed-bed reactors; plug-flow reactors; ponds; pools; quarries; reservoirs; static mixers; tanks; towers; trickle bed reactors; vats; wells—with the vessel base, siding, walls, lining, or top constructed out of one or more materials including but not limited to bitumen, cement, ceramics, clay, concrete, epoxy, fiberglass, glass, macadam, plastics, sand, sealant, soil, steels or other metals and their alloys, stone, tar, wood, and any combination thereof. In embodiments of the present invention where the chemoautotrophic microorganisms either require a corrosive growth environment and/or produce corrosive chemicals through the chemosynthetic metabolism corrosion resistant materials may be used to line the interior of the container contacting the growth medium.

Certain embodiments of the present invention will minimize material costs by using chemosynthetic vessel geometries having a low surface area to volume ratio, such as but not limited to substantially cubic, cylindrical shapes with medium aspect ratio, substantially ellipsoidal or “egg-shaped”, substantially hemispherical, or substantially spherical shapes, unless material costs are superseded by other design considerations (e.g. land footprint size). The ability to use compact reactor geometries is enabled by the absence of a light requirement for chemosynthetic reactions, in contrast to photosynthetic technologies where the surface area to volume ratio must be large to provide sufficient light exposure.

The chemoautotrophs lack of dependence on light also can allow plant designs with a much smaller footprint than photosynthetic approaches allow. In situations where the plant footprint needs to be minimized due to restricted land availability, certain embodiments of the present invention may use a long vertical shaft bioreactor system for chemoautotrophic growth and carbon capture. A bioreactor of the long vertical shaft type is described in U.S. Pat. Nos. 4,279,754, 5,645,726, 5,650,070, and 7,332,077.

Unless superseded by other considerations, certain embodiments of the present invention may advantageously minimize vessel surfaces across which high losses of water, nutrients, and/or heat may occur, or which potentially permit the introduction of invasive predators into the reactor. The ability to minimize such surfaces, in certain embodiments, is enabled by the lack of light requirements for chemosynthesis.

In certain embodiments of the present invention the chemoautotrophic microorganisms are immobilized within their growth environment. This may be accomplished using any suitable media known in the art of microbial culturing to support colonization by chemoautotrophic microorganisms including but not limited to growing the chemoautotrophs on a matrix, mesh, or membrane made from any of a wide range of natural and synthetic materials and polymers including but not limited to one or more of the following: glass wool, clay, concrete, wood fiber, inorganic oxides such as ZrO₂, Sb₂O₃, or Al₂O₃, the organic polymer polysulfone, or open-pore polyurethane foam having high specific surface area. The chemoautotrophic microorganisms in the present invention may also be grown on the surfaces of unattached objects distributed throughout the growth container as are known in the art of microbial culturing that include but are not limited to one or more of the following: beads; sand; silicates; sepiolite; glass; ceramics; small diameter plastic discs, spheres, tubes, particles, or other shapes known in the art; shredded coconut hulls; ground corn cobs; activated charcoal; granulated coal; crushed coral; sponge balls; suspended media; bits of small diameter rubber (elastomeric) polyethylene tubing; hanging strings of porous fabric, Berl saddles, Raschig rings.

Inoculation of the chemoautotrophic culture into the culture vessel, in certain embodiments, may be performed by methods including but not limited to transfer of culture from an existing chemoautotrophic culture inhabiting another carbon capture and fixation system of the present invention, or incubation from a seed stock raised in an incubator. The seed stock of chemoautotrophic strains, in certain embodiments, may be transported and stored in forms including but not limited to a powder, liquid, frozen, or freeze-dried form as well as any other suitable form, which may be readily recognized by one skilled in the art. When establishing a culture in a very large reactor it may be advantageous in certain cases to grow and establish cultures in progressively larger intermediate scale containers prior to inoculation of the full scale vessel.

The position of the process step or steps for the separation of cell mass from the process stream in the general process flow of the embodiment of the present invention illustrated in FIG. 1 is shown by the box 4. labeled “Cell Separation”.

Separation of cell mass from liquid suspension in certain embodiments of the present invention can be performed by methods known in the art of microbial culturing [Examples of cell mass harvesting techniques are given in International Patent Application No. WO08/00558, published Jan. 8, 1998; U.S. Pat. No. 5,807,722; U.S. Pat. No. 5,593,886 and U.S. Pat. No. 5,821,111.] including but not limited to one or more of the following: centrifugation; flocculation; flotation; filtration using a membranous, hollow fiber, spiral wound, or ceramic filter system; vacuum filtration; tangential flow filtration; clarification; settling; hydrocyclone. In embodiments where the cell mass is immobilized on a matrix it may be harvested by methods including but not limited to gravity sedimentation or filtration, and separated from the growth substrate by liquid shear forces.

In certain embodiments of the present invention, if an excess of cell mass has been removed from the culture, it is recycled back into the cell culture as indicated by the process arrow labeled “Recycled Cell Mass” in FIG. 1., along with fresh broth such that sufficient biomass is retained in the chemosynthetic reaction step or steps for continued optimal inorganic carbon uptake and growth or metabolic rate. The cell mass recovered by the harvesting system may be recycled back into the culture vessel using, for example, an airlift or geyser pump. In certain embodiments, the cell mass recycled back into the culture vessel has not been exposed to flocculating agents, unless those agents are non-toxic to the chemoautotrophs.

In certain embodiments of the present invention the chemoautotrophic system is maintained, using continuous influx and removal of nutrient medium and/or biomass, in substantially steady state where the cell population and environmental parameters (e.g. cell density, chemical concentrations) are targeted at a substantially constant suitable or optimal level over time. Cell densities may be monitored in certain embodiments of the present invention either by direct sampling, by a correlation of optical density to cell density, or with a particle size analyzer. The hydraulic and biomass retention times can be decoupled so as to allow independent control of both the broth chemistry and the cell density in certain embodiments. Dilution rates may be kept high enough so that the hydraulic retention time is relatively low compared to the biomass retention time, resulting in a highly replenished broth for cell growth. Dilution rates may be set at an appropriate or optimal trade-off between culture broth replenishment, and increased process costs from pumping, increased inputs, and other demands that rise with dilution rates.

To assist in the processing of the biomass product into biofuels or other useful products, the surplus microbial cells in certain embodiments of the invention are broken open following the the cell separation step using methods including but not limited to ball milling, cavitation pressure, sonication, or mechanical shearing.

The harvested biomass in certain embodiments of the present invention is dried in the process step or steps of box 7. labeled “Dryer” in the general process flow illustrated in FIG. 1.

Surplus biomass drying may be performed in certain embodiments of the present invention using technologies including but not limited to centrifugation, drum drying, evaporation, freeze drying, heating, spray drying, vacuum drying, vacuum filtration. Heat waste from the industrial source of flue gas may be used in drying the biomass in certain embodiments. In addition the chemosynthetic oxidation of electron donors is exothermic and generally produces waste heat. In certain embodiments of the present invention waste heat can be used in drying the biomass.

In certain embodiments of the invention, the biomass is further processed following drying to aid the production of biofuels or other useful chemicals through the separation of the lipid content or other targeted biochemicals from the chemoautotrophic biomass. The separation of the lipids may be performed by using nonpolar solvents to extract the lipids such as, but not limited to, hexane, cyclohexane, ethyl ether, alcohol (isopropanol, ethanol, etc.), tributyl phosphate, supercritical carbon dioxide, trioctylphosphine oxide, secondary and tertiary amines, or propane. Other useful biochemicals may be extracted in certain embodiments using solvents including but not limited to: chloroform, acetone, ethyl acetate, and tetrachloroethylene.

The broth left over following the removal of cell mass may be pumped to a system for removal of the products of chemosynthesis and/or spent nutrients which may be recycled or recovered to the extent possible, or else disposed of The position of the process step or steps for the recovery of chemical products from the process stream in the general process flow of the embodiment of present invention illustrated in FIG. 1 is indicated by the box 6. labeled “Separation of chemical products”.

Recovery and/or recycling of chemosynthetic chemical products and/or spent nutrients from the aqueous broth solution may be accomplished in certain embodiments of the present invention using equipment and techniques known in the art of process engineering, and targeted towards the chemical products of particular embodiments of the present invention, including but not limited to: solvent extraction; water extraction; distillation; fractional distillation; cementation; chemical precipitation; alkaline solution absorption; absorption or adsorption on activated carbon, ion-exchange resin or molecular sieve; modification of the solution pH and/or oxidation-reduction potential, evaporators, fractional crystallizers, solid/liquid separators, nanofiltration, and all combinations thereof.

Following the recovery of useful or valuable products from the process stream, according to certain embodiments, the removal of the waste products may be performed as indicated by the box 8. labeled “Waste removal” in FIG. 1. The remaining broth may be returned to the culture vessel along with replacement water and nutrients, if desired [see the process arrow labeled “Recycled H₂O+nutrients” in FIG. 1].

In embodiments of the present invention involving chemoautotrophic oxidization of electron donors extracted from the mineral ore, there will in certain embodimentsremain a solution of oxidized metal cations following the chemosynthetic reaction steps. A solution rich in dissolved metal cations can also result from a particularly dirty flue gas input to the process such as from a coal fired plant. In certain of these embodiment of the present invention the process stream may be stripped of metal cations by methods including but not limited to: cementation on scrap iron, steel wool, copper or zinc dust; chemical precipitation as a sulfide or hydroxide precipitate; electrowinning to plate a specific metal; absorption on activated carbon or an ion-exchange resin, modification of the solution pH and/or oxidation-reduction potential, solvent extraction. In certain embodiments of the present invention the recovered metals can be sold for an additional stream of revenue. Chemicals that are used in processes for the recovery of chemical products, the recycling of nutrients and water, and the removal of waste, may advantageously be selected in certain embodiments to have low toxicity for humans, and if exposed to the process stream that is recycled back into the growth container, low toxicity for the chemoautotrophs being used.

In certain embodiments of the present invention there is an acid co-product of chemosynthesis. Neutralization of acid in the broth can be accomplished in certain embodiments by the addition of bases including but not limited to: limestone, lime, sodium hydroxide, ammonia, caustic potash, magnesium oxide, iron oxide. In certain embodiments, the base may be produced from a carbon dioxide emission-free source such as naturally occurring basic minerals including but not limited to calcium oxide, magnesium oxide, iron oxide, iron ore, olivine containing a metal oxide, serpentine containing a metal oxide, ultramafic deposits containing metal oxides, and underground basic saline aquifers.

In addition to carbon dioxide captured through the chemosynthetic fixation of carbon, additional carbon dioxide can be captured and converted to carbonates or biominerals through the catalytic action of chemoautotrophic microorganisms in certain embodiments of the present invention. For embodiments of the invention that augment the carbon captured through chemosynthesis with biocatalyzed mineral carbon sequestration, the use of chemoautotrophic microorganisms capable of withstanding a high pH solution where carbon dioxide is thermodynamically favored to precipitate as carbonate may be advantageous in certain cases. Any carbonate or biomineral precipitate produced may be removed periodically or continuously from the system using, for example, solid/liquid separation techniques known in the art of process engineering.

An additional feature of certain embodiments of the present invention relates to the uses of chemical products generated through the chemo synthetic carbon capture and fixation process of certain embodiments of the invention. The chemical products of certain embodiments of the present invention can be applied to uses including but not limited to one or more of the following: as biofuel; as feedstock for the production of biofuels; in the production of fertilizers; as a leaching agent for the chemical extraction of metals in mining or bioremediation; as chemicals reagents in industrial or mining processes.

An additional feature of certain embodiments of the present invention relates to the uses of biochemicals or biomass produced through the chemosynthetic process step or steps of certain embodiments of the present invention. Uses of the biomass product include but are not limited to: as a biomass fuel for combustion in particular as a fuel to be co-fired with fossil fuels such as coal in pulverized coal powered generation units; as a carbon source for large scale fermentations to produce produce various chemicals including but not limited to commercial enzymes, antibiotics, amino acids, vitamins, bioplastics, glycerol, or 1,3-propanediol; as a nutrient source for the growth of other microbes or organisms; as feed for animals including but not limited to cattle, sheep, chickens, pigs, or fish; as feed stock for alcohol or other biofuel fermentation and/or gasification and liquefaction processes including but not limited to direct liquefaction, Fisher Tropsch processes, methanol synthesis, pyrolysis, transesterification, or microbial syngas conversions, for the production of liquid fuel; as feed stock for methane or biogas production; as fertilizer; as raw material for manufacturing or chemical processes such as but not limited to the production of biodegradable/biocompatible plastics; as sources of pharmaceutical, medicinal or nutritional substances; soil additives and soil stabilizers.

An additional feature of certain embodiments of the present invention relates to the optimization of chemoautotrophic organisms for carbon dioxide capture, carbon fixation into organic compounds, and the production of other valuable chemical co-products. This optimization can occur through or including methods known in the art of artificial breeding including but not limited to accelerated mutagenesis (e.g. using ultraviolet light or chemical treatments), genetic engineering or modification, hybridization, synthetic biology or traditional selective breeding. For embodiments of the present invention utilizing a consortium of chemoautotrophs, the community can be enriched with desirable organisms using methods known in the art of microbiology through growth in the presence of target electron donors, acceptors, and environmental conditions.

An additional feature of certain embodiments of the present invention relates to modifying biochemical pathways in chemoautotrophs for the production of targeted organic compounds. This modification can be accomplished, for example, by manipulating the growth environment, or through methods known in the art of artificial breeding including but not limited to accelerated mutagenesis (e.g. using ultraviolet light or chemical treatments), genetic engineering or modification, hybridization, synthetic biology or traditional selective breeding. The organic compounds produced through the modification may include but are not limited to: biofuels including but not limited to biodiesel or renewable diesel, ethanol, gasoline, long chain hydrocarbons, methane and pseudovegetable oil produced from biological reactions in vivo; or organic compounds or biomass optimized as a feedstock for biofuel and/or liquid fuel production through chemical processes.

In order to give specific examples of the overall biological and chemical process for using chemoautotrophic microorganisms to capture CO₂ and produce biomass and other useful co-products, a number of process flow diagrams &scribing various embodiments of the present invention are now described. These specific examples should not be construed as limiting the present invention in any way and are provided for the sole purpose of illustration.

FIG. 2 is process flow diagram for an exemplary embodiment of the present invention for the capture of CO₂ by hydrogen oxidizing chemoautotrophs and production of ethanol. A carbon dioxide rich flue gas is captured from an emission source such as a power plant, refinery, or cement producer. The flue gas is then compressed and pumped into cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. Hydrogen electron donor is added continuously to the growth broth along with other nutrients required for chemoautotrophic growth and maintenance that are pumped into the digester. In certain embodiments, the hydrogen source is a carbon dioxide emission-free process. This could be electrolytic or thermochemical processes powered by energy technologies including but not limited to photovoltaics, solar thermal, wind power, hydroelectric, nuclear, geothermal, enhanced geothermal, ocean thermal, ocean wave power, tidal power. Carbon dioxide serves as an electron acceptor in the chemosynthetic reaction. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the ethanol product is distilled and put through a molecular sieve to produce anhydrous ethanol using standard techniques known in the art of distillation. The broth left over after distillation is then subjected to any desired additional waste removal treatments which depends on the source of flue gas. The remaining water and nutrients are then pumped back into the digesters.

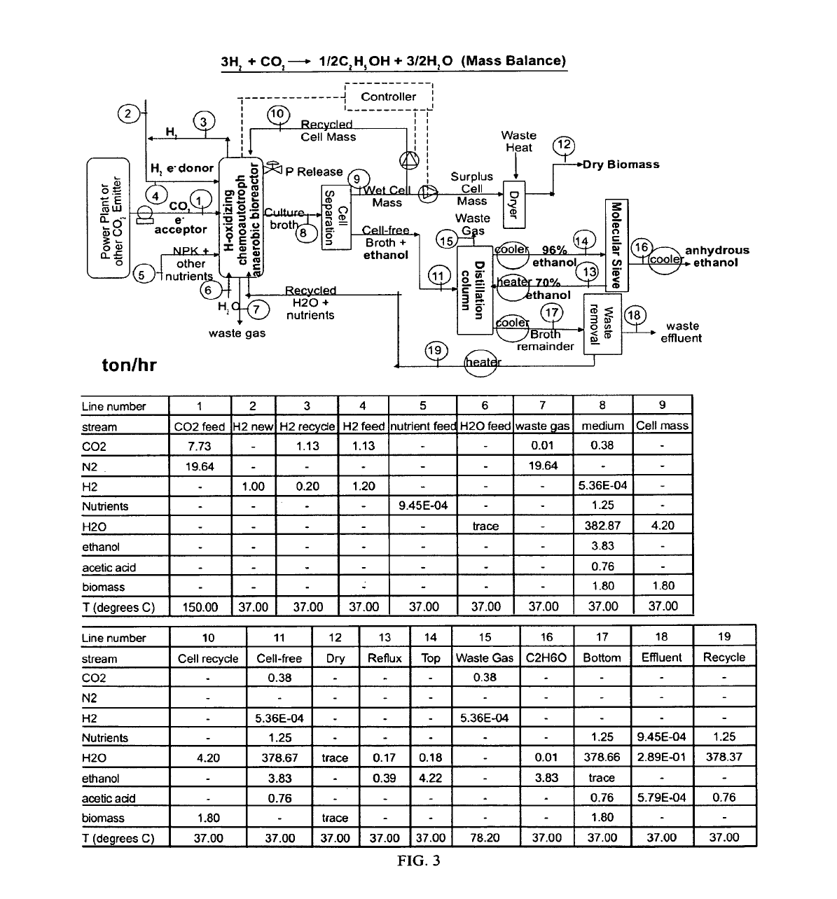

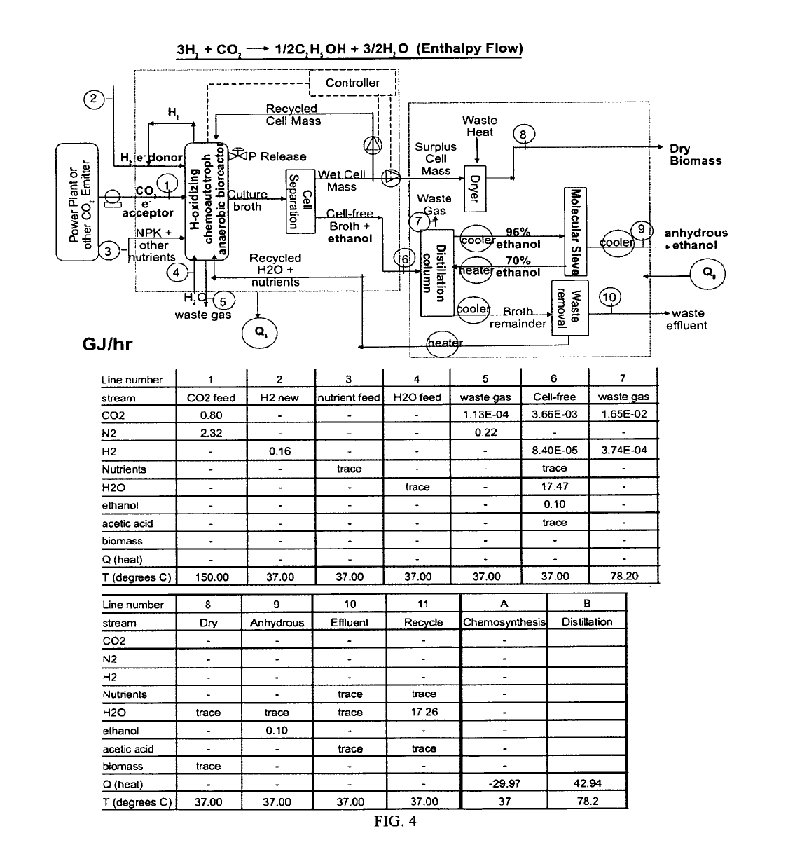

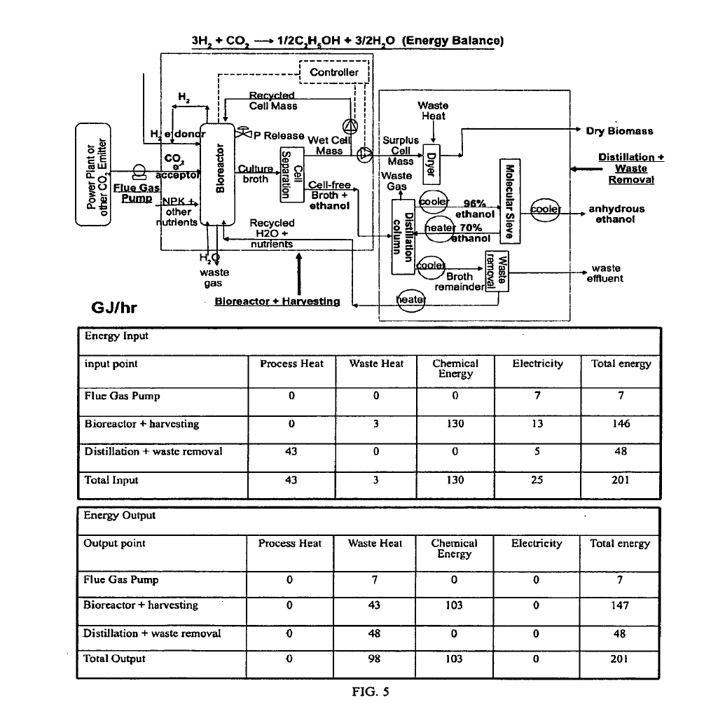

A process model is given in FIGS. 3, 4 and 5 for the embodiment of FIG. 2. The mass balance, enthalpy flow, energy balance, and plant economics have been calculated for this [R. K. Sinnott, Chemical Engineering Design volume 6, 4^{th} ed. (Elsevier Butterworth-Heinemann, Oxford, 2005)] preferred embodiment for the present invention. The model was developed using established results in the scientific literature for the H₂ oxidizing acetogens and for the process steps known from the art of chemical engineering. The inputs for the model regarding microorganism performance taken from the scientific literature [Gaddy, James L., et al. “Methods for increasing the production of ethanol from microbial fermentation”. U.S. Pat. No. 7,285,402. Oct. 23 2007; Lewis, Randy S., et al. “Indirect or direct fermentation of biomass to fuel alcohol”. US Patent Application 20070275447. Nov. 29 2007; Heiskanen, H., Virkajarvi, I., Viikari, L., 2007: The effect of syngas composition on the growth and product formation of Butyribacterium methylotrophicum. 41: 362-367] for acetogenic microorganisms were as follows: 1) stoichiometry of chemosynthetic reaction producing ethanol: 3H₂+CO₂→0.5C₂H₅OH+1.5 H₂O; 2) conversion of H₂ each pass through bioreactor: 83%; 3) stoichiometry of acetic acid side reaction: 2H₂+CO₂→0.5C₂H₅OH+H₂O; 4) Cell growth rate in plateau phase steady state ˜0; 5) percent of fixed carbon going to ethanol during steady state: 99.99%; 6) growth medium concentration of ethanol at steady state: 10 grams/liter; 7) ethanol productivity at steady state: 10 grams/liter/day; 8) concentration of acetic acid at steady state: 2 grams/liter; 9) cell mass concentration at steady state: 1.5 grams/liter. The mass balance indicates that 1 ton of ethanol will be produced for every 2 tons of CO₂ pumped into the system. This amounts to over 150 gallons of ethanol produced per ton of CO₂ intake. The energy balance indicates that for every 1 GJ of H₂ chemical energy input there is 0.8 GJ of ethanol chemical energy out, i.e. the chemical conversion is expected to be around 80% efficient. Overall efficiency of ethanol production from H₂ and CO₂ including electric power and process heat is predicted with the model to be about 50%.

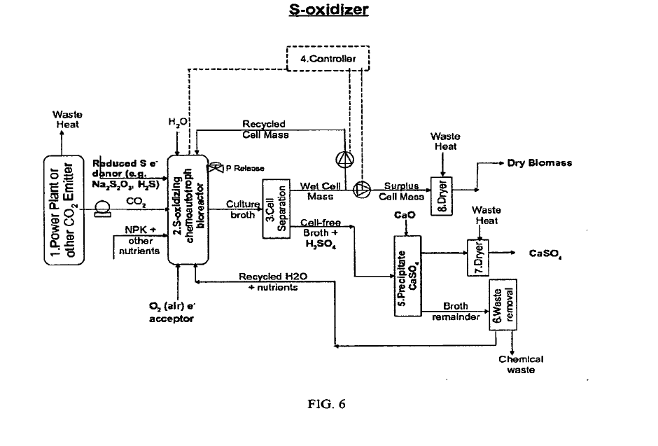

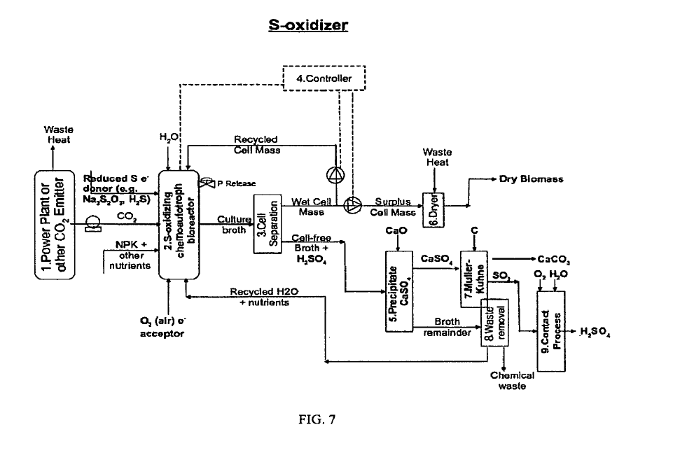

FIG. 6 is process flow diagram for an exemplary embodiment involving the capture of CO₂ by sulfur oxidizing chemoautotrophs and production of biomass and gypsum. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophila, Thiobacillus hydrothermalis, Thiomicrospira sp. strain CVO, Thiobacillus neapolitanus, Arcobacter sp. strain FWKO B. One or more electron donors such as but not limited to thiosulfate, hydrogen sulfide, or sulfur are added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime, precipitating out gypsum (CaSO₄). The lime may be produced in certain embodiments by a carbon dioxide emission-free process rather than through the heating of limestone. Such carbon dioxide emission-free processes include the recovery of natural sources of basic minerals including but not limited to minerals containing a metal oxide, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to dryers. The final product is dried gypsum. The broth left over after the sulfate is precipitated out is then subjected to any desired additional waste removal treatments which depends on the source of flue gas. The remaining water and nutrients are then pumped back into the digesters.

FIG. 7 is process flow diagram for an exemplary embodiment involving the capture of CO₂ by sulfur oxidizing chemoautotrophs and production of biomass and sulfuric acid and calcium carbonate via the Muller-Kuhne reaction. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophila, Thiobacillus hydrothermalis, Thiomicrospira sp. strain CVO, Thiobacillus neapolitanus, Arcobacter sp. strain FWKO B. One or more electron donors such as but not limited to thiosulfate, hydrogen sulfide, or sulfur are added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime (CaO), precipitating out gypsum (CaSO₄). The lime may be produced in certain embodiments by a carbon dioxide emission-free process rather than through the heating of limestone. Such carbon dioxide emission-free processes include the recovery of natural sources of basic minerals including but not limited to minerals containing a metal oxide, iron ore, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to kilns where the Muller-Kuhne process is carried out with the addition of coal. The net reaction for the Muller-Kuhne process is as follows 2C+4CaSO₄→2CaO+2CaCO₃+4SO₂. The produced CaCO₃ is collected and the CaO is recycled for further neutralization. The SO₂ gas produced is directed to a reactor for the contact process where sulfuric acid is produced. The broth left over after the sulfate is precipitated out is then subjected to any desired additional waste removal treatments which depends on the source of flue gas. The remaining water and nutrients are then pumped back into the digesters.

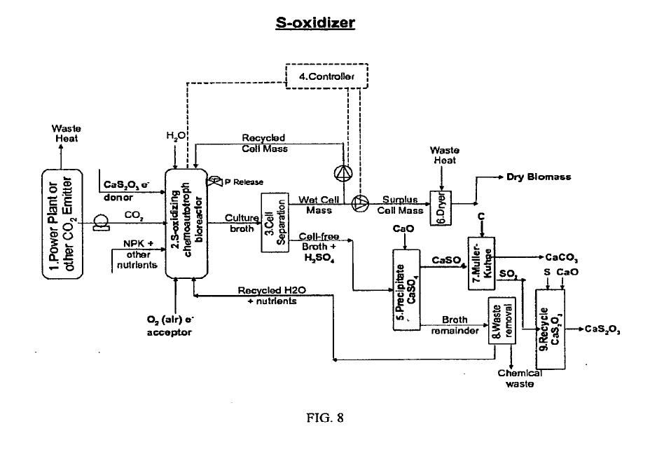

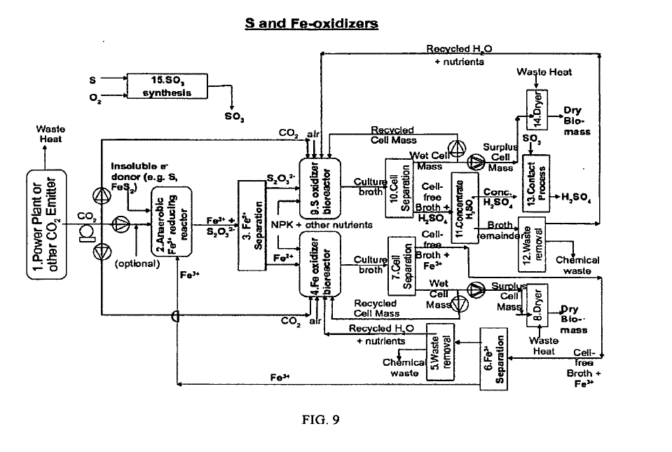

FIG. 8 is a process flow diagram for an exemplary embodiment involving the capture of CO₂ by sulfur oxidizing chemoautotrophs and production of biomass and calcium carbonate and recycling of thiosulfate electron donor via the Muller-Kuhne reaction. A carbon dioxide rich flue gas is captured from an emission source such as a power plant, refinery, or cement producer. The flue gas is then compressed and pumped into cylindrical aerobic digesters containing one or more sulfur oxidizing chemoautotrophs such as but not limited to Thiomicrospira crunogena, Thiomicrospira strain MA-3, Thiomicrospira thermophila, Thiobacillus hydrothermalis, Thiomicrospira sp. strain CVO, Thiobacillus neapolitanus, Arcobacter sp. strain FWKO B. Calcium thiosulfate is the electron donor added continuously to the growth broth along with other nutrients required for chemoautotrophic growth and air is pumped into the digester to provide oxygen as an electron acceptor. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. Cell-free broth which has passed through the cell mass removing filters is directed to vessels where the sulfuric acid produced by the chemosynthetic metabolism is neutralized with lime (CaO), precipitating out gypsum (CaSO₄). The lime may be produced in certain embodiments by a carbon dioxide emission-free process rather than through the heating of limestone. Such carbon dioxide emission-free processes include the recovery of natural sources of basic minerals including but not limited to minerals containing a metal oxide, serpentine containing a metal oxide, ultramafic deposits containing metal oxides, and underground basic saline aquifers. Alternative bases may be used for neutralization in this process including but not limited to magnesium oxide, iron oxide, or some other metal oxide. The gypsum is removed by solid-liquid separation techniques and pumped to kilns where the Muller-Kuhne process is carried out with the addition of coal. The net reaction for the Muller-Kuhne process is as follows 2C+4CaSO₄→2CaO+2CaCO₃+4SO₂. The produced CaCO₃ is collected and the CaO is recycled for further reaction. The SO₂ gas produced is directed to a reactor where it is reacted with CaO or some other metal oxide such as iron oxide, and sulfur to recycle the thiosulfate (calcium thiosulfate if CaO is used). The broth left over after the sulfate is precipitated out is then subjected to any desired additional waste removal treatments which depends on the source of flue gas. The remaining water and nutrients are then pumped back into the digesters.

FIG. 9 is process flow diagram for an exemplary embodiment involving the capture of CO₂ by sulfur and iron oxidizing chemoautotrophs and production of biomass and sulfuric acid using an insoluble source of electron donors. A carbon dioxide rich flue gas is captured from an emission source such as a power plant, refinery, or cement producer. The flue gas is then compressed and pumped into one set of cylindrical aerobic digesters containing one or more sulfur oxidizing chemoautotrophs such as but not limited to Thiomicrospira crunogena, Thiomicrospira strain MA-3, Thiomicrospira thermophila, Thiobacillus hydrothermalis, Thiomicrospira sp. strain CVO, Thiobacillus neapolitanus, Arcobacter sp. strain FWKO B, and another set of cylindrical aerobic digesters containing one or more iron oxidizing chemoautotrophs such as but not limited to Leptospirillum ferrooxidans or Thiobacillus ferrooxidans. One or more insoluble sources of electron donors such as but not limited to elemental sulfur, pyrite, or other metal sulfides are sent to a anaerobic reactor for reaction with a ferric iron solution. Optionally chemoautotrophs such as but not limited to Thiobacillus ferrooxidans and Sulfolobus sp. can be present in this reactor to help biocatalyze the attack of the insoluble electron donor source with ferric iron. A leachate of ferrous iron and thiosulfate flow out of the reactor. The ferrous iron is separated out of the process stream by precipitation. The thiosulfate solution is then flowed into the S-oxidizer digesters and the ferrous iron is pumped into the Fe-oxidizer digesters as the electron donor for each type of chemoautotroph respectively. Air and other nutrients required for chemoautotrophic growth are also pumped into the digesters. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the S-oxidizer process stream the cell-free broth which has passed through the cell mass removing filters is directed to sulfuric acid recovery systems such employed in the refinery or distillery industries where the sulfuric acid product of chemosynthetic metabolism is concentrated. This sulfuric acid concentrate is then concentrated further using the contact process to give a concentrated sulfuric acid product. The broth left over after the sulfate and sulfuric acid have been removed is then subjected to any desired additional waste removal treatments which depends on the source of flue gas. In the Fe-oxidizer process stream the cell-free broth which has passed through the cell mass removing filters is then stripped of ferric iron by precipitation. This ferric iron is then sent back for further reaction with the insoluble source of electron donors (e.g. S, FeS₂). The remaining water and nutrients in both process streams are then pumped back into their respective digesters.

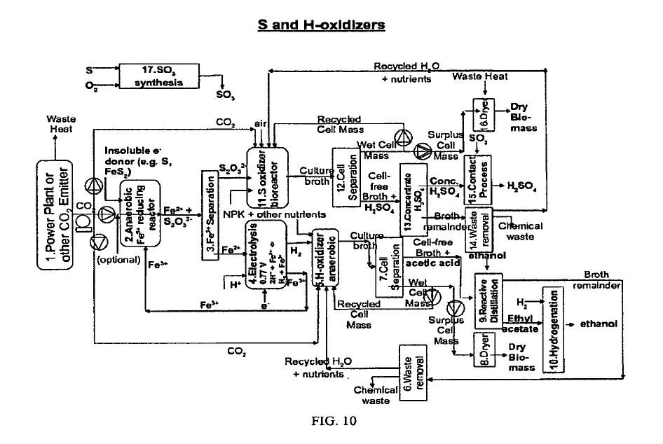

FIG. 10 is a process flow diagram for an exemplary embodiment involving the capture of CO₂ by sulfur and hydrogen oxidizing chemoautotrophs and production of biomass, sulfuric acid, and ethanol using an insoluble source of electron donors. A carbon dioxide rich flue gas is captured from an emission source such as a power plant, refinery, or cement producer. The flue gas is then compressed and pumped into one set of cylindrical aerobic digesters containing one or more sulfur oxidizing chemoautotrophs such as but not limited to Thiomicrospira crunogena, Thiomicrospira strain MA-3, Thiomicrospira thermophila, Thiobacillus hydrothermalis, Thiomicrospira sp. strain CVO, Thiobacillus neapolitanus, Arcobacter sp. strain FWKO B, and another set of cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. One or more insoluble sources of electron donors such as but not limited to elemental sulfur, pyrite, or other metal sulfides are sent to an anaerobic reactor for reaction with a ferric iron solution. Optionally chemoautotrophs such as but not limited to Thiobacillus ferrooxidans and Sulfolobus sp. can be present in this reactor to help biocatalyze the attack of the insoluble electron donor source with ferric iron. A leachate of ferrous iron and thiosulfate flow out of the reactor. The ferrous iron is separated out of the process stream by precipitation. The thiosulfate solution is then flowed into the S-oxidizer digesters as an electron donor and the ferrous iron is pumped into an anaerobic electrolysis reactor. In the electrolysis reactor hydrogen gas is formed by the electrochemical reaction 2H⁺+Fe²⁺→H₂+Fe³⁺. The open cell voltage for this reaction is 0.77 V which is substantially lower than the open cell voltage for the electrolysis of water (1.23 V). Furthermore the kinetics of the oxidation of ferrous iron to ferric iron is much simpler than that for the reduction of oxygen in water to oxygen gas, hence the overvoltage for the iron reaction is lower. These factors combined provides an energy savings for the production of hydrogen gas by using ferrous iron compared to electrolysis of water. The hydrogen produced is fed into the H-oxidizer digesters as the electron donor. The other nutrients required for chemoautotrophic growth are also pumped into the digesters. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the S-oxidizer process stream the cell-free broth which has passed through the cell mass removing filters is directed to sulfuric acid recovery systems such as employed in the refinery and distillation industries where the sulfuric acid product of chemosynthetic metabolism is concentrated. This sulfuric acid concentrate is then concentrated further using the contact process to give a concentrated sulfuric acid product. The broth left over after the sulfate and sulfuric acid have been removed is then subjected to any desired additional waste removal treatments which depends on the source of flue gas. In the H-oxidizer process stream the cell-free broth which has passed through the cell mass removing filters is directed to vessels where the acetic acid produced is reacted with ethanol to produce ethyl acetate which is removed from solution by reactive distillation. The ethyl acetate is converted to ethanol by hydrogenation. Part, e.g. half, of the ethanol is recycled for further reaction in the reactive distillation process. The other part is put through a molecular sieve which separates anhydrous ethanol by adsorbtion from dilute ethanol. The anhydrous ethanol is then collected and the dilute ethanol is returned for further reaction in the reactive distillation step. The broth left over after the acetic acid is reactively distilled out is then subjected to any desired additional waste removal treatments which depends on the source of flue gas. The remaining water and nutrients in both process streams are then pumped back into their respective digesters.

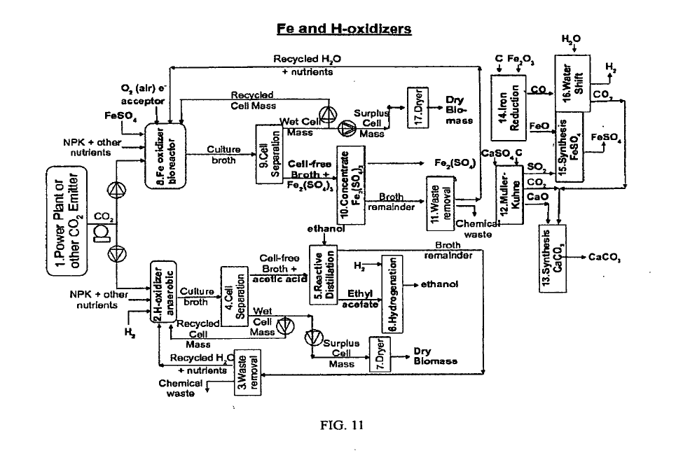

FIG. 11 is process flow diagram for an exemplary embodiment involving the capture of CO₂ by iron and hydrogen oxidizing chemoautotrophs and production of biomass, ferric sulfate, calcium carbonate and ethanol using coal or another hydrocarbon as the energy input for the production of electron donors without the release of gaseous CO₂. A carbon dioxide rich flue gas is captured from an emission source such as a power plant, refinery, or cement producer. The flue gas is then compressed and pumped into one set of cylindrical aerobic digesters containing one or more iron oxidizing chemoautotrophs such as but not limited to Leptospirillum ferrooxidans or Thiobacillus ferrooxidans, and another set of cylindrical anaerobic digesters containing one or more hydrogen oxidizing acetogenic chemoautotrophs such as but not limited to Acetoanaerobium noterae, Acetobacterium woodii, Acetogenium kivui, Butyribacterium methylotrophicum, Butyribacterium rettgeri, Clostridium aceticum, Clostridium acetobutylicum, Clostridium acidi-urici, Clostridium autoethanogenum, Clostridium carboxidivorans, Clostridium formicoaceticum, Clostridium kluyveri, Clostridium ljungdahlii, Clostridium thermoaceticum, Clostridium thermoautotrophicum, Clostridium thermohydrosulfuricum, Clostridium thermosaccharolyticum, Clostridium thermocellum, Eubacterium limosum, Peptostreptococcus productus. Hydrogen gas produced by the water shift reaction is fed into the H-oxidizer digesters as the electron donor. Ferrous sulfate synthesized through the reaction of ferrous oxide (FeO), sulfur dioxide and oxygen is pumped into the Fe-oxidizer digesters as the electron donor. The other nutrients required for chemoautotrophic growth are also pumped into the digesters for each respective type of chemoautotroph. The culture broth is continuously removed from the digesters and flowed through membrane filters to separate the cell mass from the broth. The cell mass is then either recycled back into the digesters or pumped to driers depending upon the cell density in the digesters which is monitored by a controller. Cell mass directed to the dryers is then centrifuged and dried with evaporation. The dry biomass product is collected from the dryers. In the Fe-oxidizer process stream the cell-free broth which has passed through the cell mass removing filters is directed to ferric sulfate recovery systems such as employed in the steel industry where the ferric sulfate product of chemosynthetic metabolism is concentrated into a salable product. The broth left over after the sulfate has been removed is then subjected to any desired additional waste removal treatments which depends on the source of flue gas. In the H-oxidizer process stream the cell-free broth which has passed through the cell mass removing filters is directed to vessels where the acetic acid produced is reacted with ethanol to produce ethyl acetate which is removed from solution by reactive distillation. The ethyl acetate is converted to ethanol by hydrogenation. Part, e.g. half, of the ethanol is recycled for further reaction in the reactive distillation process. The other part of the ethanol is put through a molecular sieve which separates anhydrous ethanol by adsorbtion from dilute ethanol. The anhydrous ethanol is then collected and the dilute ethanol is returned for further reaction in the reactive distillation step. The broth left over after the acetic acid is reactively distilled out is then subjected to any desired additional waste removal treatments which depends on the source of flue gas. The remaining water and nutrients in both process streams are then pumped back into their respective digesters. Both the hydrogen gas and ferrous sulfate electron donors are ultimately generated through the oxidation of coal or some other hydrocarbon. The oxidation drives two reactions that occur in parallel, one is the reduction of iron ore (Fe₂O₃) to ferrous oxide (FeO) accompanied by the release of carbon monoxide which is water shifted to produce hydrogen gas and carbon dioxide, the other is the reduction of gypsum (CaSO₄) to sulfur dioxide and quicklime accompanied by the release of carbon dioxide. The carbon dioxide from both process streams is reacted with the quicklime to produce calcium carbonate. In parallel with the production of calcium carbonate is the production of ferrous sulfate through the reaction of ferrous oxide with sulfur dioxide and oxygen.

It should be noted that in all of the previously described embodiments with a sulfuric acid product the sulfuric acid may alternatively be neutralized, in certain embodiments with a base that is not a carbonate (so as to not release carbon dioxide in the acid base reaction) and this carbonate may be produced by a carbon dioxide emission-free process. Such bases include but are not limited to natural basic minerals containing a metal oxide, serpentine containing a metal oxide, ultramafic deposits containing metal oxides, underground basic saline aquifers, and naturally occurring calcium oxide, magnesium oxide, iron oxide, or some other metal oxide.

The metal sulfate which results from the acid-base reaction may be recovered from the process stream and preferably refined into a salable product, while the water produced by the acid-base reaction may be recycled back into the chemosynthesis reactors.

The following example is intended to illustrate certain features or advantages of at least one embodiment of the present invention, but do not exemplify the full scope of the invention.

### EXAMPLE

A specific working example is provided to demonstrate the carbon capture and fixation capabilities of chemoautotrophic microorganisms that play a central part in the overall carbon capture and fixation process of the present invention.

Tests were performed on the sulfur-oxidizing chemoautotroph Thiomicrospira crunogena ATCC #35932 acquired as a freeze dried culture from American Type Culture Collection (ATCC). The organisms were grown on the recommended ATCC medium—the #1422 broth. This broth consisted of the following chemicals dissolved in 1 Liter of distilled water:

NaCl, 25.1 g; (NH₄)₂SO₄, 1.0 g; MgSO₄.7 H₂O, 1.5 g; KH₂PO₄, 0.42 g; NaHCO₃, 0.20 g; CaCl₂.2 H₂O, 0.29 g; Tris-hydrochloride buffer, 3.07 g; Na₂S₂O₃.5H₂O, 2.48 g; Visniac and Santer Trace Element Solution, 0.2 ml; 0.5% Phenol Red, 1.0 ml;

The #1422 broth was adjusted to pH 7.5 and filter-sterilized prior to innoculation.

The freeze dried culture of Thiomicrospira crunogena was rehydrated according to the procedure recommended by ATCC and transferred first to a test tube with 5 ml broth #1422and placed on a shaker. This culture was used to innoculate additional test tubes. NaOH was added as needed to maintain the pH near 7.5. Eventually the cultures were transferred from the test tube to 1 liter flasks filled with 250 ml of #1422 broth and placed in a New Brunswick Scientific Co. shake flask incubator set to 25 Celsius.

The determination of growth rate for Thiomicrospira crunogena was performed using the following procedure: 1) Three (1 litre) flasks containing 95 ml ATCC 1422 medium were innoculated with 5 ml of the above cultures diluted to an optical density ˜0.025. Optical densities were determined using a Milton Roy Spectronic 1001 Spectrophotometer; 2) Two ml samples of cultures were withdrawn from each flask from t=0 to t=48 hours at every 2 hour intervals and optical density measured. Optical density was correlated with dry weight weighing twice centrifuged and washed, 1 mL liquid broth oven dried samples in pre-weighed aluminum dishes.

From the growth curve is was found that in the exponential phase the doubling time for Thiomicrospira crunogena was one hour. This is about 4 to 6 times shorter doubling time than the fastest growth rates reported for algae in the exponential phase [Sheehan et al, 1998, “A Look Back at the U.S. Department of Energy's Aquatic Species Program—Biodiesel from Algae”]. The cell mass density present in the flask experiments when the microorganisms were in the exponential growth phase reached 0.5 g dry weight/liter, and in the plateau phase the cell mass density reached 1 g dry weight/liter. This indicates that in a continuous system that maintains the culture in the exponential growth state with continuous cell removal, these microorganisms have the potential to produce 12 g dry weight/liter/day of biomass. This is about 4-12 times faster than the highest daily rates of biomass production reported for algae [Valcent, 2007; CNN, 2008]. Furthermore, in a continuous bioreactor substantially higher cell densities should be able to be sustained in the exponential phase than what can be achieved at the flask level with T crunogena. This experiment supports the far higher rates of carbon fixation that are attainable with chemoautotrophic than photosynthetic microbes.

Specific preferred embodiments of the present invention have been described here in sufficient detail to enable those skilled in the art to practice the full scope of invention. However it is to be understood that many possible variations of the present invention, which have not been specifically described, still fall within the scope of the present invention and the appended claims. Hence these descriptions given herein are added only by way of example and are not intended to limit, in any way, the scope of this invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

What is claimed is:

1.-26. (canceled)

27. A process for the capture and conversion of carbon dioxide and/or other sources of inorganic carbon, into organic compounds, comprising:
introducing a carbon source in the form of flue gas comprising carbon dioxide and/or in the form of an aqueous solution comprising inorganic carbon into an environment in a bioreactor that is suitable for maintaining chemoautotrophic microorganisms;
introducing an electron donor that is separate from the carbon source into the environment in the bioreactor;
fixing the carbon dioxide in the flue gas and/or inorganic carbon in the aqueous solution into the organic compounds within the environment in the bioreactor via at least one chemosynthetic carbon fixing reaction utilizing chemoautotrophic microorganisms and using at least one electron donor and at least one electron acceptor; and
separating said organic compounds from a process stream produced during the fixing step,
wherein said electron donor and/or said electron acceptor are generated and/or refined from at least one inorganic chemical,
wherein said electron donor is generated separately from the carbon source and externally to the bioreactor using a renewable, alternative, or low CO₂ emission power source selected from at least one of photovoltaics, solar thermal, wind power, hydroelectric, nuclear, geothermal, enhanced geothermal, ocean thermal, ocean wave power, and tidal power,
wherein said electron donor is molecular hydrogen that is generated using said power source, through electrolysis of water, via a method using at least one of Proton Exchange Membranes (PEM), a liquid electrolyte, high-pressure electrolysis, high temperature electrolysis of steam (HTES); or through thermochemical splitting of water via a method using at least one of the iron oxide cycle, cerium (IV) oxide-cerium (III) oxide cycle, zinc zinc-oxide cycle, sulfur-iodine cycle, copper-chlorine cycle, calcium-bromine-iron cycle, hybrid sulfur cycle; or through electrolysis of hydrogen sulfide; or through thermochemical splitting of hydrogen sulfide; and
wherein the organic compounds fixed from CO₂ and/or inorganic carbon in aqueous solution comprise at least one of an organic acid, a salt of an organic acid, ethanol, and butanol, and
wherein the process for capture and conversion of carbon dioxide or inorganic carbon results in a net reduction of gaseous CO₂ released to the atmosphere.

28. A process according to claim 27, wherein said electron donor includes one or more of the following reducing agents: ammonia; ammonium; carbon monoxide; dithionite; elemental sulfur; a hydrocarbon; hydrogen; a sulfide; a sulfite; a thionate; a thionite; a transition metal and/or its sulfide; an oxide; a chalcogenide; a halide; a hydroside; an oxyhydroxide; a phosphate; a sulfate; a carbonate; and a conduction or valence band electron in a solid state electrode material.

29. A process according to claim 27, wherein said electron acceptor utilized in the chemosynthetic carbon fixing reaction comprises one or more of the following: carbon dioxide; oxygen; a nitrite; a nitrate; a transition metal ion; a sulfate; and a valence or conduction band hole in a solid state electrode material.

30. A process according to claim 27, wherein the fixing step is preceded by one or more chemical preprocessing steps in which said electron donor and/or said electron acceptor are generated and/or refined from said at least one inorganic chemical, wherein said inorganic chemical is recycled from inorganic chemicals produced during the fixing step and/or derived from waste streams from other industrial, mining, agricultural, sewage or waste generating processes.

31. A process according to claim 27, wherein the fixing step is followed by one or more process steps in which cell mass is separated from the process stream and recycled to a Attorney Docket Number: 04185.005US2/jaj reactor system in which the chemosynthetic carbon fixing reaction is performed and/or collected and processed to produce biomass in a form suitable for storage, shipping, and sale.

32. A process according to claim 27, wherein the fixing step is followed by one or more process steps in which waste products and/or impurities and/or contaminants are removed from a process stream produced during the fixing step and disposed of

33. A process according to claim 27, wherein the fixing step is followed by one or more process steps in which unused nutrients and/or process water left after removal of chemoautotrophic cell mass and/or chemical co-products of chemosynthesis and/or waste products or contaminants of the process stream produced during the fixing step are recycled back into a reactor system in which the chemosynthetic carbon fixing reaction is performed to support further chemosynthesis.

34. A process according to claim 27, wherein the chemoautotrophic microorganisms include one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Arcobacter sp.; Aureobacterium sp.; Bacillus sp.; Beggiatoa sp.; Butyribacyerium sp.; Carboxydothermus sp.; Clostridium sp.; Comamonas sp.; Dehalobacter sp.; Dehalococcoides sp.; Dehalosprillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenbacter sp.; Hydrogenomonas sp.; Leptospirillum sp.; Metallosphaera sp.; Methanobacterium sp.; Methanobrevibacter sp.; Methanococcus sp.; Methanosarcina sp.; Micrococcus sp.; Nitrobacter sp.; Nitrosococcus sp.; Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Streptomyces sp.; Sulfobacillus sp.; Sulfolobus sp.; Thiobacillus sp.; Thiomicrospira sp.; Thioploca sp.; Thiosphaera sp.; Thiothrix sp.; sulfur-oxidizer; hydrogen-oxidizers; iron-oxidizers; acetogens; methanogens; consortiums of microorganism that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater, coal seams, deep sub-surface, waste water and sewage treatment plants, geothermal power plants, sulfatara fields, and soils; and extremophiles selected from one or more of thermophiles, hyperthermophiles, acidophiles, halophiles, and psychrophiles.

35. A process according to claim 27, wherein said electron donor is generated from minerals of natural origin selected from one or more of the following: elemental Fe⁰; siderite (FeCO₃); magnetite (Fe₃O₄); pyrite or marcasite (FeS₂); pyrrhotite (Fe₍₁₋ₓ₎S (x=0 to 0.2); an iron sulfide; realgar (AsS); orpiment (As₂S₃); cobaltite (CoAsS); rhodochrosite (MnCO₃); chalcopyrite (CuFeS₂), a copper sulfide; a zinc sulfide; a lead sulfide; argentite or acanthite (Ag₂S); molybdenite (MoS₂); millerite (NiS), a nickel sulfide; antimonite (Sb₂S₃); Ga₂S₃; CuSe; cooperate (PtS); laurite (RuS₂); braggite (Pt,Pd,Ni)S; and FeCl₂.

36. A process according to claim 1, wherein said electron donor used in the chemosynthetic carbon fixing reaction is generated from pollutants or waste products selected from one or more of the following: process gas; tail gas; enhanced oil recovery vent gas; biogas; acid mine drainage; landfill leachate; landfill gas; geothermal gas; geothermal sludge or brine; metal contaminants; gangue; tailings; sulfides disulfides; one or more of methyl and dimethyl mercaptan and ethyl mercaptan; carbonyl sulfide; carbon disulfide; alkanesulfonates dialkyl sulfides; thiosulfate; thiofurans; thiocyanates; isothiocyanates; thioureas; thiols; thiophenols; thioethers; thiophene; dibenzothiophene; tetrathionate; dithioite; thionate; dialkyl disulfides; sulfones; sulfoxides; sulfolanes; sulfonic acid; dimethylsulfoniopropionate; sulfonic esters; hydrogen sulfide; sulfate esters; organic sulfur; and sour gases.

37. A process according to claim 27, wherein delivery of reducing equivalents from the electron donor to the chemoautotrophic microorganisms for the chemosynthetic reaction during the fixing step is kinetically and/or thermodynamically enhanced by one or more of introduction of hydrogen storage materials into the environment in the bioreactor in the form of a solid support media for microbial growth that facilitates bringing absorbed or adsorbed hydrogen electron donors into close proximity with the chemoautotrophic organisms; introduction of electron mediators selected from one or more of cytochromes, formate methyl-viologen, NAD⁺/NADH, neutral red (NR), and quinones to help transfer reducing power from poorly soluble electron donor comprising H₂ gas or electrons in solid state electrode materials into chemoautotrophic culture media in the bioreactor; and introduction of electrode materials in the form of a solid growth support media directly into the environment in the bioreactor that facilitates bringing solid state electrons into close proximity with the chemoautotrophic microorganisms.

38. A process according to claim 27, wherein said electron donor used in the chemosynthetic carbon fixing reaction is generated within or recycled to the environment in the bioreactor through non- or low-carbon dioxide emitting chemical reactions with hydrocarbons selected from one or more of thermochemical reduction of sulfate reaction (TSR) and the Muller-Kuhne reaction for the production of hydrogen sulfide or reduced sulfur; and methane reforming-like reactions utilizing metal oxides in place of water, the metal oxides selected from one or more of iron oxide, calcium oxide, and magnesium oxide; and wherein the hydrocarbon is reacted to form solid carbonate with little or no emissions of carbon dioxide gas along with hydrogen electron donor product.

39. A process according to claim 27, wherein said at least one chemosynthetic carbon fixing reaction is performed by chemoautotrophic microorganisms that have been improved, optimized or engineered for the fixation of carbon dioxide and/or other forms of inorganic carbon and the production of organic compounds through methods including one or more of the following: accelerated mutagenesis, genetic engineering or modification hybridization, synthetic biology and traditional selective breeding.

40. A process according to claim 27, wherein organic and/or inorganic chemical products are recovered from chemoautotrophic growth medium of the at least one chemosynthetic carbon fixing reaction, and wherein the organic and/or inorganic chemical product are useful as biofuels or as feedstock for biofuel production; in the production of fertilizers; as leaching agents for the chemical extraction of metals in mining or bioremediation, and/or as chemicals reagents in industrial or mining processes.

41. A process according to claim 27, wherein biomass and/or biochemicals are produced by the at least one chemosynthetic carbon fixing reaction, and wherein the biomass and/or biochemical are useful as a biomass fuel for combustion; as a fuel to be co-fired with fossil fuels; as a carbon source for large scale fermentations to product at least one of commercial enzymes, antibiotics, amino acids, vitamins, bioplastics, glycerol, and 1,3-propanediol; as a nutrient source for the growth of other microbes or organisms; as feed for animals selected from cattle, sheep, chickens, pigs, and/or fish, as feed stock for biofuel fermentation and/or gasification and liquefaction processes comprising direct liquefaction, Fisher Tropsch processes, methanol synthesis, pyrolysis, transesterification, or microbial syngas conversions for the production of liquid fuel; as feed stock for methane or biogas production; as fertilizer; as raw material for manufacturing or chemical processes; as sources of pharmaceutical, medicinal or nutritional substances; and/or as soil additives and soil stabilizers.

42. A process according to claim 27, wherein said bioreactor comprises and/or is formed at least in part by a microbial culture apparatus selected from: an airlift reactor; a biological scrubber column; a bubble column; a continuous stirred tank reactor; a counter-current, upflow, expanded-bed reactor; a digestor for a sewage and/or waste water treatment or bioremediation system; one or more filters; a fluidized bed reactor; a gas lift fermenter; an immobilized cell reactor; a membrane biofilm reactor; a mine shaft; a Pachuca tank; a packed-bed reactor; a plug-flow reactor; a static mixer; a tank; a trickle bed reactor; a vat; and/or a vertical shaft bioreactor.

43. A process according to claim 27, further comprising prior to the fixing step, a step of reacting carbon dioxide with minerals to form a carbonate or bicarbonate product, which is then used in the fixing step.

44. A process according to claim 1, wherein the inorganic carbon in the aqueous solution comprises carbonate ion, bicarbonate ion, and/or carbon dioxide.

45. A process according to claim 44, wherein the aqueous solution comprises seawater.

46. A process according to claim 45, wherein the inorganic carbon in the aqueous solution is a carbonate ion and/or a bicarbonate ion that is derived from a carbonate mineral.

47. A process according to claim 42, wherein the apparatus comprises a vessel having a base, siding, walls, lining, and top, at least one of the base, siding, walls, lining, and top being constructed out of a material selected from bitumen, cement, ceramics, clay, concrete, epoxy, fiberglass, glass, macadam, plastics, sand, sealant, soil, steels, non-steel metals, metal alloys, stone, tar, wood, and combinations thereof.

48. A process according to claim 27, wherein the chemosynthetic carbon fixing reaction utilizes electron donors and/or electron acceptors introduced from at least one inorganic source or waste source.

49. A process according to claim 43, wherein the minerals comprise oxides or hydroxides.

50. A process according to claim 27, comprising fixing the carbon dioxide and/or inorganic carbon into the organic compounds via at least one chemosynthetic carbon fixing reaction within a reactor system, wherein the electron donor utilized in the chemosynthetic carbon fixing reaction is produced via a non-biological process in the reactor system.

51. A process according to claim 27, wherein the chemosynthetic microorganisms are obligate anaerobes.

52. A process according to claim 51, wherein the chemosynthetic microorganisms are acetogens.
